(19) United States
(12) Patent Application Publication
(10) Pub. No.: US 2024/0384355 A1
(43) Pub. Date: Nov. 21, 2024

(54) NEXT GENERATION SEQUENCING AND ARTIFICIAL INTELLIGENCE-BASED APPROACHES FOR IMPROVED CANCER DIAGNOSTICS AND THERAPEUTIC TREATMENT SELECTION

(71) Applicant: JSR Life Sciences, LLC, Durham, NC (US)

(72) Inventors: Emanuel Schmid-Siegert, Lausanne (CH); Romain Groux, Lausanne (CH); Thierry Schuepbach, Echandens (CH); Bonnie Chen, Suzhou (CN); Ioannis Xenarios, Lausanne (CH); Alaaddin Bulak Arpat, La Croix (Lutry) (CH); Huan Tian, Suzhou (CN); Xingxia Wu, Suzhou (CN)

(21) Appl. No.: 18/690,065

(22) PCT Filed: Sep. 8, 2022
(86) PCT No.: PCT/US22/42899

§ 371 Date: Mar. 7, 2024

Related U.S. Application Data
(60) Provisional application No. 63/241,813, filed on Sep. 8, 2021.

(51) Int. Cl. C12Q 1/6886; C12Q 1/6841; C12Q 1/6869

(52) U.S. Cl. CPC C12Q 1/6886; C12Q 1/6841; C12Q 1/6869; C12Q 2600/106; C12Q 2600/118; C12Q 2600/156

(57) ABSTRACT

Provided herein are methods for identifying and predicting the progression of a cancerous state in an asymptomatic subject or a subject suffering from cancer; and compositions and kits related thereto. Methods include identifying from a sequencing of a sample at least one gene fusion, non-gene fusion, genomic alteration, transcriptomic alteration or neotranscript, wherein presence of said gene fusion, non-gene fusion, genomic alteration, transcriptomic alteration or neotranscript indicates one of an increased risk of developing cancer, a subject as a candidate for cancer therapy, or an increased risk of resistant or metastatic cancer.

### RELATED APPLICATIONS

This application claims the benefit of U.S. Provisional Application No. 63/241,813, filed on Sep. 8, 2021. The entire teachings of the above applications are incorporated herein by reference.

In most countries, cancer is diagnosed at an increased frequency each year, because of changing population demographics and aging. In 2015, cancer rose to over 40,000 diagnosed cases yearly in Switzerland, with a 5-year survival rate under 60% overall (Swiss Federal Statistics Office, 2019). Breast and prostate cancers are among the most frequent cancers to be diagnosed, pancreatic cancer shows the lowest survival rate among the 10 most frequent cancer types, with a 5-year survival of approximately 10%. Despite research efforts, the evolution, severity, and response to available treatments remain difficult to evaluate using current pathological histology and molecular analysis. Therefore, a lack of pathological response remains typically around 50% for most therapies, yielding decreased prognosis and quality of life for the patient, while increasing costs.

In the case of pancreatic cancer, with the exception of surgical resection of asymptomatic early-stage tumors, the disease is still largely considered incurable. Pancreatic cancer is most often diagnosed at later metastatic stages, where surgery has only limited efficacy, and an efficient and specific pharmacological treatment is still lacking. Consequently, non-specific and non-curative chemo- and radiotherapies are often used to increase life expectancy, with severe consequences for the patients' quality of life. Aggressive forms of pancreatic cancers, such as pancreatic ductal adenocarcinoma (PDCA), are most frequently diagnosed at late stages, when no longer resectable, after spreading to neighboring tissues and/or forming metastases. Early stages of PDCA are mostly asymptomatic and current serum-based assays cannot differentiate indolent pancreatitis from mucinous pancreatic adenocarcinoma (Carmicheal et al., 2019). Current analysis of the mutational load, such as mutations or upregulation of KRAS and EGFR, is not sufficient to diagnose and properly classify pancreatic cancer types, as these are common to many cancers. At present, there is no efficient, sensitive, and non-invasive asymptomatic diagnostic approach that can be used routinely. Late stage PDCA are notoriously difficult to treat, as surgery often proves inefficient in the long term because of relapse, and because specific therapeutic treatments are lacking. Chemo- and/or radiotherapies are often used as palliative care in adjuvant therapies, which will not be curative in most cases. Therefore, there is a clear and unmet need for a non-invasive, sensitive, and inexpensive diagnostic method to detect cancers, e.g., pancreatic cancer, at an early stage; while curable by surgical resection, and for developing and applying more efficient and specific therapies (Carmicheal et al., 2019). Similarly, breast and prostate cancers are other examples of tumors difficult to diagnose properly in terms of progression and drug response (Davidson et al., 2019; Pondé et al., 2019).

### SUMMARY OF THE INVENTION

Provided herein are methods for predicting the likelihood of progression of an asymptomatic subject to a cancerous state, comprising the steps of:
(a) sequencing at least part of the subject's genome in a sample from said subject, and
(b) identifying from the sequencing of said sample at least one gene fusion, non-gene fusion, genomic alteration, transcriptomic alteration or neotranscript, wherein presence of said gene fusion, non-gene fusion, genomic alteration, transcriptomic alteration or neotranscript indicates an increased risk of developing cancer.

In certain aspects, provided herein are methods for identifying an asymptomatic subject for personalized cancer therapy, comprising the steps of:
(a) sequencing at least part of the subject's genome in a sample from said subject,
(b) identifying from the sequencing of said sample at least one gene fusion, non-gene fusion, genomic alteration, transcriptomic alteration or neotranscript, wherein presence of said gene fusion, non-gene fusion, genomic alteration, transcriptomic alteration or neotranscript identifies the subject as a candidate for personalized cancer therapy, and
(c) initiating said therapy and/or monitoring administration of the therapy to the subject.

Aspects of the invention, as provided herein, include methods for predicting tumor response or resistance in a subject suffering from cancer, comprising the steps of:
(a) sequencing at least part of the genome of one or more cells in a sample of the subject;
(b) identifying in said sample at least one gene fusion, non-gene fusion, genomic alteration, transcriptomic alteration or neotranscript, wherein presence of said gene fusion, non-gene fusion, genomic alteration, transcriptomic alteration or neotranscript indicates an increased risk resistant cancer.

In certain aspects, provided herein are methods for predicting the likelihood of metastasis in a subject suffering from cancer, comprising the steps of:
(a) sequencing at least part of the genome of one or more cells in a sample of the subject;
(b) identifying in said sample at least one gene fusion, non-gene fusion, genomic alteration, transcriptomic alteration or neotranscript, wherein presence of said gene fusion, non-gene fusion, genomic alteration, transcriptomic alteration, or neotranscript indicates an increased risk of metastasis.

Also provided herein are methods comprising performing a bioassay to detect at least one gene fusion, non-gene fusion, genomic alteration, transcriptomic alteration, or neotranscript comprising or transcribed from at least one of the genes set forth in Table 1 in a sample from a subject, receiving the results of the bioassay into a computer system, processing the results to determine an output, presenting the output on a readable medium, wherein the output identifies therapeutic options recommended for the subject based on the presence or absence of the at least one gene fusion, non-gene fusion, genomic alteration, transcriptomic alteration, or neotranscript, wherein the sample is a liquid or tissue biopsy.

In some aspects of the invention, provided herein are cancer diagnostic kits comprising at least one reagent allowing the detection of at least one gene fusion or non-gene fusion in a sample from a subject, wherein said fusion comprises or is transcribed from at least one of the genes set forth in Table 1.

In certain aspects, provided herein are compositions comprising at least one of the following: (a) a detection probe comprising an oligonucleotide sequence that hybridizes to a junction of a gene fusion, non-gene fusion, genomic alteration, transcriptomic alteration or neotranscript comprising at least one sequence selected from SEQ ID Nos. 1-65; (b) a first labeled probe comprising an oligonucleotide sequence that hybridizes to a 5′ portion of a gene fusion, non-gene fusion, genomic alteration, transcriptomic alteration, or neotranscript comprising or transcribed from at least one sequence selected from SEQ ID Nos. 1-65, and a second labeled probe comprising an oligonucleotide sequence that hybridizes to the corresponding 3′ portion of the gene fusion, non-gene fusion, genomic alteration, transcriptomic alteration, or neotranscript; (c) a first amplification oligonucleotide comprising a sequence that hybridizes to a 5′ portion of a gene fusion, non-gene fusion, genomic alteration, transcriptomic alteration, or neotranscript comprising or transcribed from at least one sequence selected from SEQ ID Nos. 1-65, and a second amplification oligonucleotide comprising a sequence that hybridizes to the corresponding 3′ portion of the gene fusion, non-gene fusion, genomic alteration, transcriptomic alteration, or neotranscript; (d) an antibody that specifically binds to an amino acid sequence encoded by at least one sequence selected from SEQ ID Nos. 1-65 and (e) an in situ hybridization probe for detecting a gene fusion, non-gene fusion, genomic alteration, transcriptomic alteration or neotranscript comprising at least one sequence selected from SEQ ID Nos. 1-65.

### BRIEF DESCRIPTION OF THE DRAWINGS

FIG. 1 shows the outcome for pancreatic cancer patients. Three arms of patient outcome were established over years. The resectable arm, which comprises around 10-20% of patients, was subjected either to Resection or Neoadjuvant treatment (Neoadj. Tx) prior to resection. When an Adjuvant treatment (Adj Tx) was applied following Resection (76-92% of the patients follow this path), survival ranges from 20.1 to 23.6 months, while a 16.9-20.2 months survival was observed for patients not submitted to the Adjuvant treatment. Neoadjuvant treatment prior to resection allows better survival of patients within the Resectable group (73.6% of the patient follow that route), leading to an average of 23.3 months survival. The second arm, concerning 30-40% of the patients, consists of a Neoadjuvant treatment followed by a resection for 33.2% of the cases allows a 20.5 months survival, whereas the non-resected patients have a 10.2 months life expectancy. When palliative treatments were provided, instead of Neoadjuvant treatments, mean survival ranges from 6-11 months. Finally, Metastatic pancreatic cancer represents 50-60% of the patients who had a mean survival of 5-9 months under palliative treatments.

FIG. 2 shows the discovery engine pipeline enabling the detection and characterization of novel fusions. The pipeline was assembled by several pieces of software to transform the RNA and DNA sequencing data originating for different sequencing technologies into a usable and evidenced based fusion. Step 1 consisted of evaluating, quality controlling, and filtering of the obtained raw sequences. Subsequently, the read origin (mouse/human or unknown) was performed under Step 2, then branching into a Step 2a, which was a mapping strategy which consists in indexing each sequence on a human genome reference catalog (e.g. Refseq). Sequences found to bridge two genomic locations were detected and classified as either a known fusion (i.e., already established in the scientific literature or in diagnostic practice) or a novel fusion criteria if being previously unknown. For genomic or RNA sequences that were not recognized as known or novel fusions, a next step was performed to assemble these sequences to be able to detect novel gene and/or non-gene fusion sequences. Step2b consisted of a de-novo assembly-based approach to enable a comprehensive assessment of all fusions. This was performed on both long and short sequencing reads, and used to classify fusions amongst the different organisms (mouse and human) present in the sample. When performed with RNA sequences, a pan-transcriptome database was constructed that served as a basis for the discovery of novel neotranscript fusions.

FIG. 3 shows scoring and prioritization of neotranscripts and genomic fusions, and classification feature impact. By using a machine learning approach on features derived from the fusion identification, a prioritization scheme was identified that enabled sorting and assessment of the likelihood of occurrence for each candidate fusion. The performance of the method was determined as 94%, as evaluated with the harmonic F1 score, indicating excellent performance. The measures used to assess and evaluate each fusion were benchmarked on known fusion/transcripts, either spiked in the raw sequence dataset in silico, or experimentally introduced into the RNA/DNA preparations at different concentrations. The features used were the gene distance between the two partners, Fusion Score (derived from several internal metrics of the sequencing reads), the open reading frame (ORF) length (if it existed), the length of the fusion, the identification of a Split Pair and Split Read supporting fusion point, the origin or start site (for any coding gene product, when occurring), the Coverage (representing an estimation of frequency of the fusion in the sample), a measure of Junction in Orf describing the quality of the junction, the level of expression per transcript, and the fusion confidence (being a derived metric of confidence). All features were scored from high to low, therefore enabling a selection of assessment to be automatically applied to each fusion. The type of features that have a positive or negative effect on the predictive score value are illustrated for identified neotranscript/genome fusions.

FIG. 4 shows use of distinct sequencing technologies for discovering novel genomic/transcript fusions. Different sequencing technologies have distinct advantages and short-comings. This was documented by using single or combined sequencing datasets obtained from PDX pancreatic cancers. The neotranscripts/fusions obtained from the indicated datasets are depicted by each column, and the number of candidate fusions identified are on the y-axis. The 433 selected validated fusions consist of the sum of the candidate fusions shown by the last two columns.

FIG. 5 shows validation of the PDX pancreas cancer fusion sequence dataset using known genomic alterations. The mutations depicted on the X axis were analyzed on the EGFR (left panel) and KRAS (right panel) coding sequences, using the datasets obtained from the 136 pancreatic cancer PDX models illustrated on the Y axis. The large fraction of the tumor samples that contains KRAS mutations that are typical of pancreatic cancer is illustrated by the box.

FIG. 6 shows a heatmap of the candidate genomic fusions and occurrence among pancreatic cancer samples. Clustering of the occurrence of neotranscripts/fusions in 136 PDX pancreatic cancer samples is depicted on the top row in relation to several classifications, ranging from ethnicity (Asian/Western), subtype (adenocarcinoma, adenosquamous carcinoma, mucinous adenocarcinoma, neuroendocrine adenocarcinoma rosis, and unclear), biopsy site (diaphragm, liver, lymph node, omentum, pancreas, paracentesis, pleural, stomach and unknown) and pathology grade (moderate, moderate to poorly, poorly, unclear, well). The histogram on the right side represents the number of PDX pancreatic cancers harboring a particular neotranscript/fusion. The classification is based on fusions that were either highly frequent or rare (observed only in 1-2 PDX pancreatic model).

FIG. 7 shows a fraction of pancreatic cancer neotranscripts/fusions shared with other cancer types. The possible occurrence of 433 neotranscript/fusions identified in PDX cancer samples was assessed in various PDX cancer samples relative to their occurrence in pancreatic cancer models. Each pancreatic cancer neotranscript/fusion is represented by a line, whereas the cancer types evaluated are represented as columns (MK=Merkel carcinoma, AM=Acute Myeloid Leukemia, MC=Metastatic Carcinoma, XX=Unknown, PR=Prostate, AD=Adrenal Cancer, MU=Mullerian, UT=Uterine, KI=Kidney, GL-Gall Bladder, CV=Cervical, BL=Bladder, OV=Ovarian, BR=Breast, HN=Head and Neck, ES=Esophageal, LU=Lung, Li=Liver, CC-Colon, GA=Gastrointestinal, CR=Colorectal, PA=Pancreatic, Al=Acute Lymphoblastic, LY=Lymphoma, SA=Sarcoma, ME=Melanoma, BN=Brain). The fraction (0 to 100%) of the samples containing the fusions is depicted from light grey (occurrence in 100% of the cancer types, e.g. top lines) to black (occurrence in 1% or less of the cancer types, e.g. bottom lines). Approximately 47 neotranscripts/fusions were found to occur exclusively in pancreatic cancers, except for one which was also present in lung cancers.

FIG. 8: shows a heatmap and classification of pancreatic cancer cell growth and doubling rates. The doubling growth rate, i.e., the time required to double the volume of the grafted cancer tissue, was measured for a subset of the PDX models consisting of 48 samples, which displayed doubling time ranging from 5 to 30 days. A comparison of the doubling growth rate to the neotranscript/fusion content was assessed, considering an arbitrary <10 days threshold for fast growers and >10 days for slow growers. This allowed the classification of some of the undetermined samples as predicted aggressive and fast growers (double dashed line).

FIG. 9: shows the PCA of 400 most differentially regulated genes in PDX PDAC (1) and GTEX pancreatic patients (2+3). Highlighted in white (3) is a subset of GTEX patients which carried gene fusions from the candidate fusions for PDAC.

FIG. 10: shows the number of total expressed genes (>=1 TPM) found for each sample in different cohorts.

FIG. 11: shows the number of fusion events in pancreatic samples. FIG. 11A depicts the number of total gene-fusion events found for each sample in different cohorts and FIG. 11B depicts the number of high-confident events per sample. High confidence is defined by multiple read support, precision and additional evidence.

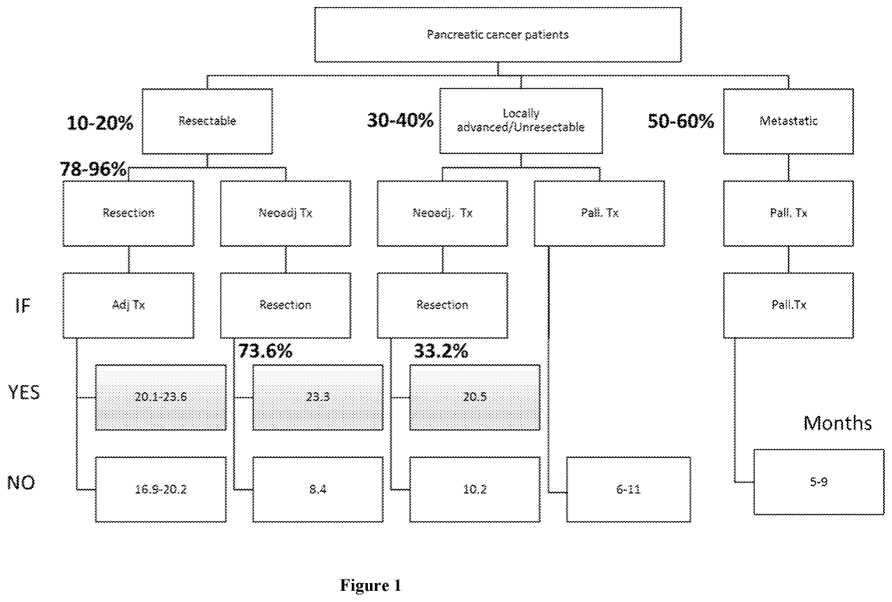

### DETAILED DESCRIPTION OF THE INVENTION

Large scale genomic studies of human tumors propagated by xenotransplantation into immunocompromised mice, termed patient-derived xenograft (PDX) models, have shown promising results in terms of prediction of drug response in precision medicine and its translation to several cancer patients. Such models have also proven useful for establishing the mechanisms of resistance, thus proving to be more informative than cell line models (Gao et al., 2015). However, some potentially relevant markers such as copy number variations and large chromosomal alterations were not captured by such studies. This is exemplified by amplification of the p53 regulator MDM4 or the phosphoglycerate dehydrogenase (PHGDH) genes which were not found in breast cancer or pancreatic ductal adenocarcinoma (PDAC). They may be due to limited PDX sample numbers, lack of sufficient next-generation sequencing (NGS) depth, and/or insufficient data mining and analysis (Gao et al., 2015; Kim et al., 2019).

Despite progress, efficient diagnostic tools for frequent or particularly lethal cancers (e.g., prostate and breast tumors, and such as pancreatic cancer) often fail to predict the best therapeutic approach, and therapies remain inefficient for a proportion of affected patients. So far, the development of in vitro diagnostic and therapeutic approaches are limited by the lack of large collections of tumor samples associated to reliable clinical database, and by the need for comprehensive molecular data sets and analytical tools capable of handling big datasets. Thus, there are clear unmet needs in terms of datasets, as well as approaches, allowing early asymptomatic diagnosis as well as efficient and specific therapeutic approaches in oncology.

Disclosed herein is analysis of the genome and transcriptome of one of the largest collections of cancer patient-derived xenotransplant (PDX) tumor samples, following their transplantation and propagation into murine models. DNA and RNA next-generation sequencing (NGS) datasets were collected and mined in relation to the patient clinical data and tumor properties. Genomic and transcriptomic alterations linked to cancer progression were identified and characterized using artificial intelligence (AI) based models. Focusing on frequent cancers that lack efficient diagnosis and treatment (e.g. pancreas cancer) or that remain difficult to diagnose and prognose accurately (e.g. breast and prostate cancers), the NGS data were mined and correlated to the tumor type and patient clinical data, as well as to the tumor pathological response to therapeutic treatments. A specific set of genomic and transcript alterations that can describe various tumor types when analyzed by artificial intelligence-based models can be identified, so as to distinguish distinct cancers from one another, as well as from their cognate healthy tissues. Furthermore, subsets of these markers can be identified to predict the aggressiveness of given tumor types, as can be analyzed from clinical blood samples or tumor biopsies. Thus, a first outcome made possible by the identification of such cancer markers is a more sensitive and specific early diagnosis of tumor occurrence using clinical samples obtained from patients. An improved prognosis of tumor evolution may also be achieved, as may be needed to evaluate whether a surgical intervention is suited and to predict the tumor response or resistance to available therapeutics, using such comprehensive NGS and AI-based in vitro diagnostic (IVD) approach.

In addition, provided herein are specific sets of genomic and transcriptomic markers and novel AI based algorithms that constitute tools that can be applied to provide a diagnosis of cancer occurrence, tumor aggressiveness, and response or resistance to available therapeutics. Such tools allow an early asymptomatic diagnosis of cancer, to better distinguish various cancer types, and to prognose more accurately their evolution. Such markers also provide more reliable predictions of the patient response to available therapeutics, and thereby allow selection of the most appropriate therapy for each patient. The available therapeutics are in part covered by the clinical annotation of each PDX sample analyzed. Overall, the outcome of the tools and methods disclosed herein are to provide improved strategies for in vitro diagnosis (IVD), precision medicine, and personalized therapies in the oncology field.

Thus, provided herein are methods for predicting the likelihood of progression of an asymptomatic subject to a cancerous state, comprising the steps of:
(a) sequencing at least part of the subject's genome in a sample from said subject, and
(b) identifying from the sequencing of said sample at least one gene fusion, non-gene fusion, genomic alteration, transcriptomic alteration or neotranscript, wherein presence of said gene fusion, non-gene fusion, genomic alteration, transcriptomic alteration or neotranscript indicates an increased risk of developing cancer.

In certain aspects, provided herein are methods for identifying an asymptomatic subject for personalized cancer therapy, comprising the steps of:
(a) sequencing at least part of the subject's genome in a sample from said subject,
(b) identifying from the sequencing of said sample at least one gene fusion, non-gene fusion, genomic alteration, transcriptomic alteration or neotranscript, wherein presence of said gene fusion, non-gene fusion, genomic alteration, transcriptomic alteration or neotranscript identifies the subject as a candidate for personalized cancer therapy, and
(c) initiating said therapy and/or monitoring administration of the therapy to the subject.

Aspects of the invention, as provided herein, include methods for predicting tumor response or resistance in a subject suffering from cancer, comprising the steps of:
(a) sequencing at least part of the genome of one or more cells in a sample of the subject;
(b) identifying in said sample at least one gene fusion, non-gene fusion, genomic alteration, transcriptomic alteration or neotranscript, wherein presence of said gene fusion, non-gene fusion, genomic alteration, transcriptomic alteration or neotranscript indicates an increased risk resistant cancer.

In certain aspects, provided herein are methods for predicting the likelihood of metastasis in a subject suffering from cancer, comprising the steps of:
(a) sequencing at least part of the genome of one or more cells in a sample of the subject;
(b) identifying in said sample at least one gene fusion, non-gene fusion, genomic alteration, transcriptomic alteration or neotranscript, wherein presence of said gene fusion, non-gene fusion, genomic alteration, transcriptomic alteration, or neotranscript indicates an increased risk of metastasis.

### Fusions

Fusions in general are produced through interchromosomal and intrachromosomal rearrangements (e.g., translocations, deletions, inversions, duplications, and the like) and may result in a plurality of combinations of coding and non-coding sequence. DNA or RNA sequence fusions disclosed herein consist of DNA or RNA sequences which are fused together in cancer cells while they are disjoint in sets of normal reference cells. Such fusions can be further classified as either gene fusions when encompassing coding, e.g., protein-coding sequences, whereas non-gene fusions encompass non-coding sequences, e.g., DNA sequences that do not code for amino acids, and may include, as non-limiting examples, DNA lying outside and/or between genes on the chromosome; introns; and DNA elements that play a role in the regulation of gene expression. Some gene fusions have coding potentials and may produce in-frame protein coding sequences or non-coding regulatory RNAs with new or altered functions, which may be linked to cancer occurrence or progression. For example, and without being bound by any particular theory or methodology, such fusions may result in proteins and/or regulatory RNAs that modulate cancer-associated genes or gene products. It will be appreciated by those of skill in the art that such fusions may be intrachromosomal (e.g., fusions arising from rearrangements occurring within a chromosome as are known in the art, such as duplications/amplifications, insertions, deletions, inversions, and the like) or interchromosomal (e.g., fusions arising from rearrangements occurring between two or more chromosomes, such as translocations or more complex structural genome variations as are known in the art, including but not limited to complex chromosomal rearrangements such as insertion-translocations, inversions associated with copy number variation, translocations affecting more than 2 chromosomes, and combinations thereof). Accordingly, in some embodiments, the fusions disclosed herein may comprise one or more interchromosomal fusions, one or more intrachromosomal fusions, or any combination thereof. In some such embodiments, the fusions contemplated and disclosed herein may comprise coding and/or non-coding DNA sequences.

Some fusions termed known fusions were previously observed to occur in particular cancer cells (Tembe et al., 2014 or Haas et al., 2019) whereas the unknown fusions or novel fusions identified herein were not previously reported to our knowledge. When transcribed in the cell, gene fusions that constitute novel fusions can be identified or detected as neotranscript fusions.

Candidate fusions have several features that are captured computationally, such as the fusion point and the gene fusion partners (if those are coding genes), or by other annotation if they possess distinct features such as encoding regulatory RNA (e.g., lnRNA). A Score was developed to assess the predicted accuracy of predicted fusions, so as to assess whether the unknown fusions may be trusted and occur frequently in similar types of cancers.

Examplary fusions are disclosed herein by an NGSAI-ID identifier of the form NGSAI-NEOTX-1 to NGSAI-NEOTX-69. (See Table 1)

TABLE 1
Identified Fusions

| NGSAI_ID | Gene1 | Gene2 |
|---|---|---|
| NGSAI_NEOTX_1 | SPPL2A | USP8 |

ATGAGAGTTGGAGAAAAAACTACTGGCTTGGTGGAACGATCAGGAATAACTTTTC
CACTCTGAGGAGATTGTTGCTCATGGTTTGTACTTTCAGATTTTATTCTATGCTCTT
CAGGCAATTTGACTGCTGGTTTTTTAGTACGATCAATCTTGAAGTTGGGCAACTTC
AGTGTTTTAATTAAATTCAGACAGAAAGCAATCCCCAAGATATCCTGTAAAATCCA
AGCCCACCTGTCTTCATTTCGAAACACAGCCCAAACAACAGCTACTGCTATGCACA
GTCCAGAGAGAAAAATAAGTC (SEQ ID NO. 1)

| | | |
|---|---|---|
| NGSAI_NEOTX_2 | FBRSL1 | LOC105370091 |

GAGGGTACCGAGATTGGCCGTCGGCTGGCAGGCGCCCAGGAGAGCCGGTGGCGT
GAGCTCCAAGCCTGAAGGCAGGGGAGGACCCACGTCCCAGCCCGAATCGAGCAG
TGTGTGTGAACACTCCCTGCCTCGGCCTTTCTGTCCTACTCAGGCCTCGCCGGCGC
CCCAGGCAGTCGCCCCTAGTCCCGGGGCCGGAGCCGGGCTGCATGGACGCGGGCG
TGGAGCGCGAGCCCCGGGTGGCCCTGGCCCGTCCAGGCGACCCCTCTCCCCGCGT
GCCCTGCTCAGCCGGAGCTCGGGCCGG (SEQ ID NO. 2)

| | | |
|---|---|---|
| NGSAI_NEOTX_3 | MIRLET7BHG | PAK4 |

AAGTTGGACGGCGCGGAGATCTCCACCCGCTTCTTCCTCTTCCCAAACATGGTGCC
GGGGACTCGGTGCGGCCTGCACACACCTGGTCTGATGCTGGTGGGACAGAAGTGC
CCTCAGGCAGGTGACCACTCCTCTAGGGGCTTCGGGTTACTCATCCGAGGTGCCGG
AGGATGGAGGCGTCTTCTCCAAAGCCAGGAAGTGAAAATGACGTCCCTGGGCCCA
GCCGGTCACCCGGGTGGGGGAGGAGGGCAGGTCCCGCCGGCCAGCAGGCTGCCC
GGTGCCAGCCCCAGCTATGGGCCCA (SEQ ID NO. 3)

| | | |
|---|---|---|
| NGSAI_NEOTX_4 | PAK4 | PRR34-AS1 |

CTCGAAGTTGGACGGCGCGGAGATCTCCACCCGCTTCTTCCTCTTCCCAAACATGG
TGCCGGGGACTCGGTGCGGCCTGGTCTGATGCTGGTGGGACAGAAGTGCCCTCAG
GCAGGTGACCACTCCTCTAGGGGCTTCGGGTTACTCATCTTTTCACGGGAAGTGAC
CCTCCTCCCCATGGGTCAATAAGTTAACGCCAAATCGCGGCAAAACGGCGAATTC
CATCTCTGAGGCTCTAGAAGCTCAATCTTCTGGGCCCCTGGCTCCTGGCCTCGGGT
CCTGCTGGTGCCCAGGTCGCCCG (SEQ ID NO. 4)

| | | |
|---|---|---|
| NGSAI_NEOTX_5 | LOC400682 | ZNF85 |

AGCATTGACAATAGGCACAATATAGTTTTGCATTGGTGTCTGTGAATTTGATAGAG
CAAACACTTCTTCAAGTTGTTTTTTTTGTTTTGTTTTGTTTTTTTGTTTTTTTTTTGAG
ACAGCATTTTGCTCTTGTTGCCCAAGCTGGAGTGCAGTGTCATGATCTTGGCTCAC
AGCAACCTCCGCCTCCCGGGTTCAAGTGATGCTCATTTCTAGGCTTCCAGGAGGAC
CTGGCGTCTTAGCTGGGGATCTCCCAATACCTGCAGGTCACAGGGCCACAGAGGC
TGGGCCCCTAGGAGAAGAG (SEQ ID NO. 5)

| | | |
|---|---|---|
| NGSAI_NEOTX_6 | DOCK1 | FAM196A |

GGCCTGCTGTCAGCTGCTCAGCCACATCCTGGAGGTGCTGTACAGGAAGGACGTG
GGGCCAACCCAGAGGCACGTCCAGATTATCATGGAGAAACTTCTCCGGACCGTGA
ACCGAACCGTCATTTCCATGGGACGAGATTCTGAACTCATTGCTTTCATATCGGAC
GGGGCAAGACACAGCTAATTGTGACACATGCAGGAACAGTGCATGTATTATCTAT
AGTGTGGAGCTGGATTTTAAGCAGCAAGAAGACAAACTCCAGCCGGTTCTAAGAA
AACTCCACCCTATTGAGGAAACTCA (SEQ ID NO. 6)

| | | |
|---|---|---|
| NGSAI_NEOTX_7 | CLVS1 | RAB2A |

CAATTCCAACATGGTCATTATGCTTATTGGAAATAAAAGTGATTTAGAATCTAGAA
GAGAAGTAAAAAAAGAAGAAGGTGAAGCTTTTGCACGAGAACATGGACTCATCTT
CATGGAAACGTCTGCTAAGACTGCTTCCAATGTAGAAGAGGGCCTAATCAAGGGA
ATGGAAGATGAGGAGAATGAAGGCTCTGGGAATTTATTTTCATCGTGGACCGGAC
TTTTTATCAGCCAGGAAAAAGGTGTGGTGGCTCACGCCTGTAATCCTAGCACTTTG
GGAGGCCGAGCTGGGAGGATTTCT (SEQ ID NO. 7)

| | | |
|---|---|---|
| NGSAI_NEOTX_8 | CPAMD8 | NWD1 |

TGAGGTCGACGTGTGTGTGACCTCTCTTCATCTGGCCGTGACCCCCAGCATGGTCC
CCCTTGGTCGCCTGCTGGTCTTCTACGTCAGGGAGAATGGAGAAGGGGTCGCCGA
CAGCCTTCAGTTTGCAGTCGAGACCTTCTTCGAAAACCAGGTCGTTGATCTGAGGT
GGGGTATTCGGAACATTGAAGCCACTGACCACTTGACCACAGAACTCTGCTTGGA
GGAGGTTGACCGGTGTTGGAAAACATCCATAGGGCCAGCTTTTGTTGCCCTCATCG
GTGATCAGTACGG (SEQ ID NO. 8)

| | | |
|---|---|---|
| NGSAI_NEOTX_9 | TMEM254-AS1 | TMEM254-AS1 |

CTGGCCAATATGGTAAAACCCCATCTCTACTAAAAATACAAAAATTATCCGGGCG
TGGTGGCACGCTCCTGTAATCTCAGCTACTCAGGAGGCTGAGGACTACAGGTGCC
CGCCGCCACGGCTAGCTAATTTTTTTTTGTATTTTTTAGTAGAGACAGTGTTTCACC
GTCTCTACTAAAGATCAAGGATGGTCTTGATCTCCTGACCTGGTGATCCACCCACC
TCAGCCTCCCACAGTGCTG (SEQ ID NO. 9)

| | | |
|---|---|---|
| NGSAI_NEOTX_10 | LOC105375130 | PLAGL2 |

AAGTGAAGTGCCAATGTGAAATTTCGGGAACACCTTTCTCAAATGGGGAGAAGCT
GAGGCCTCACAGCCTCCCGCAACCAGAGCAGAGACCATATAGCTGCCCTCAGCTG
CACTGTGGCAAGGCTTTTGCTTCCAAATACAAGCTGTATAGGATAAACTAAACAG
GCCTCAAGAATGTGACCTCCCACGCTCCTCCATGAACAGCTCTCTCCCTGCGTCCC
AGCAACCAAAGACACTTGTTGATTTGGGAAAAACCCAGAGGAAGGATTCTGTCTG
GATTTTCTGGTACCACTGACGCATT (SEQ ID NO. 10)

| | | |
|---|---|---|
| NGSAI_NEOTX_11 | ANKRD27 | CPAMD8 |

AGCAGTTGCTGATGGAGATCTAGAAATGGTGCGTTACCTGTTGGAATGGACAGAG
GAGGACCTGGAGGATGCGGAGGACACTGTCAGTGCAGCAGACCCCGAATTCTGTC
ACCCGTTGTGCCAGTGCCCCAAGTGTGCCCCAGCTCAGAAGGAAACGGGACTGGT
GGTGATGACCGACCGAGTGAGCCTGAACCACCGGCAGGACGGTGGCCTCTACACC
GATGAGGCTGTCCCCGCTTTCCAGCCCCACACAGGGAGCCTGGTGGCAGTGGCTC
CTTCCAGGCACCCCCCCAGAACAGAG (SEQ ID NO. 11)

| | | |
|---|---|---|
| NGSAI_NEOTX_12 | IL20RB | TRIM74 |

CTCACTGCAACCTCCACCTCCTGGGTTCTAGCGTTTCTCCTGCCTCAGCCTCCCAAG
TAGCTGGGATTACAGGAATGTGCCACCATGCTTGGCTAATTTTGTATTTTTAGTAG
AGACAGTGTTTCACTATGTTGGCCAGGCTGGTCTCGAACTCCTGACCTCAGGTGAT
CCGCCCACCTTGGCCTCCCAAAGTGCTGGGATTACAGGAGTGAGCCACCACGCCC
AGCCTCCGTTGTCCTCATTTAGACTTTCCTGGGTTATAGGCACTTTTGACTTCCTGG
GGTCCTTCTTCAGTTAAAAA (SEQ ID NO. 12)

| | | |
|---|---|---|
| NGSAI_NEOTX_13 | CHS.26712.1 | ZNF829 |

CGGGCATGGTGGCGTGCACCTGTAGTCCCAGCTACTGAGGAGGCTGAGGCAGGAG
AATTGCTTGAACTCGGGAGGTCAAGGTTGTAGTGAGCCGAGATCGCACCACTGCA
CTCCAGCACTCCAGCCTGGGTGACAGCAAGACTCTGTCTCTGAACACAGGCCTCTA
GTCAGCTCTCTATCAACCATCCAGGGCTCTTTTCCTTGTTCCAATAAGGAGATCAC
AGCTGGCTTAGAATTGGAAAGTCCCACTGAAACCAGGTTGCTGAAATTCTCCAAC
ATCACTTCTTTGTATAAATTCATC (SEQ ID NO. 13)

| | | |
|---|---|---|
| NGSAI_NEOTX_14 | NA | ZNF431 |

CCTTTGAGTCTCCAGAGTCCACTGTGTCATTCTTATGCTTTTGCATCCTCATAGCTT
AGCTCCCGCTTATGAGTGAAAACATACGATGTTTGGTTTTCCATTCCTGAGTTACTT
CACTTAGAACCCTCCTTGACCTATCTCAGTGCTGGGATTACAGGCGTGAGCCACAC
CTGGCTGCCTTTTAACTGTTCTGATAAGCAAACTCTACAGTTAAAACCAATTTTTGT
GTGCACTAAAAATACCAACTTCCTCATCAAAATCTACAAAGTACC (SEQ ID NO. 14)

| | | |
|---|---|---|
| NGSAI_NEOTX_15 | RFX8 | RNF149 |

TCCAAAAGCAACAAGTGAAACAGATGCCAGGAGCCAAAACTATCTCTGTGGCAGA
GGGTCATGGCTTTGCTTACAGCAGTGAGAAGAAGATTCCATCTCAGCTGGAAGCT
GGCGGCCAATTTGGTAAACTCTTCTTTGCTTCTGCTTGTCTAGCAATGCCGTATTTT
CTCCTGCATCTCCTTTAAAGCTGGGATCACACTGTGGCTCAGATTCAGCAGGGGAG
GCTGATGGTGGACTGCTCTCATCACTTCCGTCATCATCTGGTAAAGCTAGACTCAA
ATTTGCAGCTGGATCCCTTCCA (SEQ ID NO. 15)

| | | |
|---|---|---|
| NGSAI_NEOTX_16 | KIF13B | KPNB1 |

CTGGTGTGCTCAGGGGGCCGTCCTTGTTCTGCTGCCTCTGAAGCTTCAATGGCCAA
ATCCATTTCCTCATCACAGACATTGGACCAGAATTCTATCCCTTGTAAAGCCACCT
CATCAATGTCACTTTTCATTGCTTCGATTGTGATCATTTAAGTCCAGGAATATAAC
AGGAATGTGTGTCTCCATCCCAGGTACTGGGGTCCATTCTGCTGGGGCCCCTGGAA
TACCACTGCCAGCAGAGGGGACCATCACCGCGTTCCTCTCCTCAGTTAGGGTCAAC
CGCATCTCCAGAAGCTGCGCT (SEQ ID NO. 16)

| | | |
|---|---|---|
| NGSAI_NEOTX_17 | RHPN2 | ZNF569 |

AAACTATAACAACTTAATCACAGTAGGCTATCCGTTCACCAAACCTGATGTGATTT
TCAAATTGGAGCAAGAAGAAGAACCATGGGTGATGGAGGAAGAAGTATTAAGGA
GACACTGGCAAGCATAATAAGAGTGCCACATACTCCGTGGGAATGCAGAAAACGT
ACTCCATGATCTGCTTAGCCATTGATGATGACGACAAAACTGATAAAACCAAGAA
AATCTCCAAGAAGCTTTCCTTCCTGAGTTGGGGCACCAACAAGAACAGACAG (SEQ
ID NO. 17)

| | | |
|---|---|---|
| NGSAI_NEOTX_18 | LOC105378701 | STIL |

AAACCAACTCCATTTGTCTTCCAGCTTGCACTGCGTCTTCAACAGCAGTTGTCTTA
GGGGAACAGGGCATCAGAGACTGTGCTTCCAACAAACGCTGAATCTGGTAGGATC
ATTGTGAGGCTCCAATCAGAAAGTGTCTTACACATCATACAGTAGCCCTCAGATTC
AATGTAGAAAACAGCACCAGCAAATGTAAATTAGTACAACCATTGTGGAAGACAG
TGTGG (SEQ ID NO. 18)

| | | |
|---|---|---|
| NGSAI_NEOTX_19 | GREB1L | LAMA3 |

GCCAGATACTGCTTCAGTTCCAGGGTTAACGTATCTCAGAATAACACGAAACAAG
GAGCCACTTGACTTCCCTACATTCAATGTTATTCTTACATCATTCTCTCCAAGAGTG
TGTTCACCATTTTTTCAAAAGTCTCGTCACATCTCAGAAGTGGGCTCGTGATCCCC
CACTGCAGAGACTTGCTGTACTCACTCAAGCCAAAGTACAGCTTCTCCTCG (SEQ
ID NO. 19)

| | | |
|---|---|---|
| NGSAI_NEOTX_20 | LAMC1 | LAMC2 |

GGTGCCTTCAATCTCGTTTAGCTTATTCAGGTCCACTGTATCCAGCTGCCCCAGCTG
CTCCAAGAGGTCATTAATAATGCTGAGGAGGCTAGTAACAGAGTTTTTGGCTTTTC
TGGCATTGATCTCGGCTTCTTGAGCAGCCTGTGAAGCCCATCAGATGCAGGAGGCC
GTCTAATGTGTTGAGTGTGTCTTGGATTGTAACCCCAGCGTTCTTGGCTCTGGTATC
AACCTTCTGGGCTTCTGTAATCACCATCTGTACTGCATCCATATTCGTGTCAAACTC
CAGCTCCTTCCTTTCCAG (SEQ ID NO. 20)

| | | |
|---|---|---|
| NGSAI_NEOTX_21 | CHEK1 | LOC105369526 |

TGAGCCTCGCCCCGGCAGCTTCCAAGAGAGAGCAGAGGTGCTGGAAAGGGCACA
AGAGCAGGAACTCGAGGACCTGGTTTGCATCTCAGCTCTGGCACGTCCTTGCTGGT
GACTCATTGCATAACCTCCCTGAGCCTTGGTCTTCTTGTCTGATTCATACAACTTTT
CTTCCATTGATAGCCCAACTTCTCACAAGTCTCTTTCAGGCATTGATAAGATTTGTC
TGCATCCAATTTGGTAAAGAATCGTGTCATTCTTTTGACCAACCGCTGCCAGGGGT
TCTGTGAGGATCCTGGGGTGC (SEQ ID NO. 21)

| | | |
|---|---|---|
| NGSAI_NEOTX_22 | ARHGAP32 | ME3 |

GAGGTTTAGTTTTTTTTGTTTTTTAAGTACAAGATGGAGACTGAAAGTGAGAGTAG
CACTTTAGGGGATGACAGTGTCTTCTGGTTGGAGTCTGAAGTTATAATCCAGGTGA
CTGACTGTGAAGAGGAAGAAAGGGAAGAGAAGTTCAGGGGATGGCCTTTACCCTT
GAAGAAAGGCTGCAGCTTGGAATCCACGGCCTAATCCCGCCCTGCTTTCTGAGCC
AGGACGTCCAGCTCCTCCGAATCATGAGATATTACGAGCGGCAGCAGAGTGACCT
GGACAAGTACATCATTCTCATGAC (SEQ ID NO. 22)

| | | |
|---|---|---|
| NGSAI_NEOTX_23 | GMNN | MYO6 |

GAGCTGTGGCCTTTTGCGAGGTGCTGCAGCCATAGCTACGTGCGTTCGCTACGAGG
ATTGAGCGTCTCCACCCATCTTCTGTGCTTCACCATCTACATAATGAATCCCAGTAT
GAAGCAGAAACAAGAAGAAATCAAAGAGAATATAAAGGTGGTTGTAATCTGAAG
AATAAATCTGCTCAGTCTTTGGAATATTGTGCTGAATTACTGGGTTTGGACCAAGA
TGATCTTCGAGTAAGTTTGACCACAAGAGTCATGCTAACAACAGCAGGGGGCACC
AAAGGAACAGTTATAAAGGTACC (SEQ ID NO. 23)

| | | |
|---|---|---|
| NGSAI_NEOTX_24 | CCDC134 | IRAK1 |

TCACGCCTGGCTAATTTTTGTATTTTTAGTAGAGACGGGGTTTCACCAAGCTGGCC
AAGCTGGTCTCGAACTCCCGACCTCAGGCAATCCGCCCACCTCAGCACTTTGGGA
GGCCAAGGCAGGAGGATCGCTGGAGCCCAGTAGGTCAAGACCAGCCAGGGCAAC
ATGATGAGACCCTGTCTCTGCCAAAAAATTTTTTAAACTATTAGCCTGGCGTGGTA
GCGCACGCCTGTGGTCCCAGCTGCTGGGGA (SEQ ID NO. 24)

| | | |
|---|---|---|
| NGSAI_NEOTX_25 | CHS.3009.1 | FAM120A |

TTTCCTGCCTTAGCCTCCCAAAGTGCTGGGATTACAGGTGTGAGGCACTATGCCCG
GTTCATTTATGTTTTAAAAGTCTCATATAACTAGCCGGGTGTGGTGGCTCATGCCT
ATAATCTCAGCATTTTGGGAGGCCAAGGAGAGAGAATTGCTTGAGGCAGGAGTTC
AAGACCAGCCTGGGCAATATAGTGAGACCCCTGCCTCTACAAAAAATTTTAAAAA
TTAGCCAGGTATGGTGGTGCACACCTGTAGTCCCAGCTACTCAGGAGGCTGAGGC
GGGAGGATCGCTAGAGCTGGGAGG (SEQ ID NO. 25)

| | | |
|---|---|---|
| NGSAI_NEOTX_26 | HIVEP3 | SEPHS1 |

CTATAAAATGTCCAATCACTTTCAGTTCCGTAGCAGGCTCTTCCATACTGCACACC
ATGCTTATGGCTGGAGGTCCAGTTACACATGCATGAAGGCTGCCCTGCCCACTGGT
TCCTGGAGGAGGGCGCGTCCGAGTTAAAGCCTCTTCTTAGCCTTCGGCCTTGGGAT
GGCAAACTGGTCCTGTGTTCTCTGACCCACGGATCCAGCCCCTTCTTCATGAATTA
TTCCGGCCAGGCAGGATTTTGTGCATTTTTTTCATGAACACCTGCGCGCCGGGCCG
GGGCGGCGGGAGGCGGCTTGG (SEQ ID NO. 26)

| | | |
|---|---|---|
| NGSAI_NEOTX_27 | JMJD1C | JMJD1C-AS1 |

AGAGCTGGTGGGTAAGCGGTTCCTGTGTGTGGCGGTCGGCGACGAGGCACGTTCG
GAGCGCTGGGAGAGCGGACGCGGCTGGCGAAGCTGGCGAGCGGGGGTCATCCGA
GCCGTGTCACACAGGGACAGCCGCAATCCGGACCTGGCGGTACTTTCAAACCTCT
GGTTGAAAGAAATATACCCAGTTCAGTCACTGCAGTAGAATTCCTTGTAGATAAG
CAACTGGATTTTTTAACTGAAGATAGTGCCTTTCAGCCCTACCA (SEQ ID NO. 27)

| | | |
|---|---|---|
| NGSAI_NEOTX_28 | CTSH | SMAD3 |

GGTTCAGTGCCATTTTAAATGTGTGGTTCCCATTGTTGTGGGCGTTTATCTTCCTCC
AGTTGCTGGCAAACGTCTGCAGCCTGTGGTGGTACTCCTCCGTACTGTAGGTCTTA
CGGTGCTTAGACATCCATGACTTGAAGTGAAACTTCTCCAAGTTATTATGTGCTGG
GGACATCGGATTCGGGGATAGGTTTGGAGAACCTGCGTCCATGCTGTGGTTCATCT
GGTGGTCACTGGTTTCTCCATCTTCACTCAGGTAGCCAGGGGGGGGGTCTCTGGA
ATATTGCTCTGGGGCTCGAT (SEQ ID NO. 28)

| | | |
|---|---|---|
| NGSAI_NEOTX_29 | PHIP | SH3BGRL2 |

ATCCAGTTTCAGCTTTCTACATATGGCTAGTCAGTTTTTCCAGCACCACTTATTAAA
TAGGGAATCCTTTTCCCATTGCTTGTGTTTGTCAGGTTTGTCAAAGATTAGATGGTT
GTAGATGTGTGGTGTTATTTCTGAGGCCTCTGTTCTGGATTGTTTGAGCCCACGAA
TTCAAAGCCAGTCTTGTCATATTTGCTACTGGACCCAAAGCCAAAAATTAAAAGAT
GTCCATGAGAGTCTGTGCATGCAAAATGCTGACCATCAGGAGAGCATTTGCAGTC
AAATACTGCGCCATGTCCTT (SEQ ID NO. 29)

| | | |
|---|---|---|
| NGSAI_NEOTX_30 | LOC101929831 | NT5C3B |

ACCTTCATCACCAGAGGCTTGAAGGAACCCCGCCATGTGGCAGGGCACAGGCACT
GTTCCTGGTGAACCTTGGACCACAGCATGTCAGTGCTCTAGGGATTGTCTACTCCA
GGGATTTTCTTCAAAATTTTTAAACATGGGAAGTTCAAACCTAGTGCATAGTAGGG
AGTCAGTAAGTGTTACTCACTTCTCTCCCTTCCTCTCCTGAACCACGAGCGTTAAA
AATATTTTGTAAGGATGAAACTTCCAGAACTTGTGTTCAAATAATAATTAACACGG
GCTGGGCCTTTTCCTGAGAAGC (SEQ ID NO. 30)

| | | |
|---|---|---|
| NGSAI_NEOTX_31 | LOC105374140 | U2SURP |

ACTTTAAGTAAAAAGGAACAGGAAGAATTAAAGAAAAAGGAGGATGAAAAGGCA
GCTGCTGAGATTTATGAGGAGTTTCTTGCTGCTTTTGAAGGAAGTGATGGTAATAA
AGTGAAAACATTTGTGCGAGGGGGTGTTGTTAATGCAGCTAAAGGAGCACCTGTG
GGCATCTTTCCTCAACGCCCGGACTACAAATCTCTAACACGAGTTGTTGGCTGAGG
ACAGATTCTCATGGCCGGAAACCACCACTTCCCTTGGACATGCATGCGTTGGCTGG
GTACTGG (SEQ ID NO. 31)

| | | |
|---|---|---|
| NGSAI_NEOTX_32 | SSFA2 | UBE2E3 |

CTCCGACGCTTGCCAGGAGCTGCGGCACTTGGCCCAGGCCTTCCTCCTGCGACTCG
CCACTTGCCACTCCAGTTCCTCCTCCGCCTCCGCCGACGACGACAGGGGCCGGTCC
ATGGCCGCACTGGGGGCTCCGCTACCCCAGCCGGACCCTGCAATTAGGAGGAGGA
TCAAGGGTTATTTCAGCTAGCTCCTTCTGAATTCTTTTAGCACTAGTGGATAACTTA
GCAGTGGTTTTGCTAGAGAGTTTGGTGTTTTTCTTCTGCTGGGTGGCAGAAGGTTTT
CTTTCCTCTTGTTCTTCAGG (SEQ ID NO. 32)

| | | |
|---|---|---|
| NGSAI_NEOTX_33 | LOC107985961 | RP11-796E10.1 |

TCAACTCTTATCCACACAGAAGAGCTCTCTTCCAGGGCTGCTGGTGAAAGCAGGTG
CAATCAGAGGAGCCATAAGTCACAGCGATTCTGCAGGTGAGGAGGAAATGATGCC
ATGTGGCGAGACTTGGCCTTTAAGAACTGCAAATAGAGCGGAGGAGCCAAGATGG
CCGAATAGGAACAGCTCCGGTCTACAGCTCCCAGCTTGAGTGACGCAGAAGATGG
GTGATTTCTGCATTTCCATCTGAGGTACCGGGTTCATCTCACTGAATACTGCGCTTT
T (SEQ ID NO. 33)

| | | |
|---|---|---|
| NGSAI_NEOTX_34 | LOC400958 | TET3 |

TTGCACTAGCTGTACCAACCGCCGCACGCACCAGATCTGCAAACTGCGAAAATGT
GAGGTGCTGAAGAAAAAAGTAGGGCTTCTCAAGGAGGTGGAAATAAAGGCTGGT
GAAGGAGCCGGGCCGTGGGGACAAGGAGCGGCTGTCAAGGTGCCTCAGCCTCGA
ACCTTGTGATGAGTGAGAAATCTTTCTCCCCTACGGGTGAAGGAAAGAGCCTGAG
TCTCTGCTGTGGCTGGGGACAGGAAATGCACCCACCTGCCAAGCTGCTGGTGACA
CCTGGTGGCAGCCAGGAAGCCCCAGACT (SEQ ID NO. 34)

| | | |
|---|---|---|
| NGSAI_NEOTX_35 | GATA6 | SEH1L |

GATGGGGTAACTTGCTTGGGCTGAGGTTGCAGACGTTACCCCCAACAGAAGATAG
GTAGAAATGATTCCAGTGGCCTCTTTGTATTTTCTTCATTGTTGAGTAGATTTCAGG
AAATCAGGAGGTGTTTCACAATACAGAATGATGGCCTTGCCTTCCAGCTAGCAGT
ACAATGCCAATCACCACTTTCACTTTTATCCCAGACCTTAACGCTCTGATCGCTGG
AGCAGGTTGCCATCCGCCGCCCGTGGAAGTCGAAAGAGACATCGTGGATGAGATC
CTTGTGGTCCGCCGCGATGCTGC (SEQ ID NO. 35)

| | | |
|---|---|---|
| NGSAI_NEOTX_36 | LOC105376010 | MTAP |

GCAATATGTAATGATCTGTTTGGCTGGTGGTCACTTAATTCTTCTAACCTGTTTCCT
TATCTTTGATTGTCATTCATTTTTCCTTTTACTTTTTCTTCCATTTGTGATGCTCAGC
CACAACTTGAGATTTAAAATCATCAAAAACATACTCACCTCTCTCGTTTTGGGGCA
AAACGGCTCAGCCATTGGAATATGGCACACTCCTCTGGCACAAGAATGACTTCCA
TCATAGAAGGACTGAGGTCTCATAGTGGTCCTGTCAATGAACTGATCAATAATGA
CAATATCGCCGGGCTGAATC (SEQ ID NO. 36)

| | | |
|---|---|---|
| NGSAI_NEOTX_37 | CHS.27064.2 | ZG16B |

GTGAAACCCAGTCTCTACTAAAAATACAAAAATTAGCCGGGCATGGTGGTGTGCG
CCTATAATCCCAGATACTCAGGAGGCTGAGGCAGCAGAATCACTTGAACATGAGA
CGTGGAGGTTGCAGTGAGCCAAGATTGCACTACTGCACTCCAGCCTGGGTGACAG
AGTAAGACTCTGTCTAAAGAGAGAAAGAAAGAAAAGAAAAGAAAAGAGAAAAG
AAAAGAAAAGAAAAGAAAAGAAAAGAAAAGAAAAGAAAAGAAAAGGGCCAGGT
GTGGTGGCTCACACCT (SEQ ID NO. 37)

| | | |
|---|---|---|
| NGSAI_NEOTX_38 | CDRT1 | FGD4 |

CGGGCGCAGTGGCTCATGCCTGTAATCCCAGTACTTTGGGAGGCCGATGCGGTTG
GATCATGAGGTCAGGAGATCAAGACCATCCTGGTTAACATGGTGAAACCCCGTCT
CTACTGATACTTAGGTCATAGCTCCCGCTTAGGAGAAAGTTTTCCTCCTCACACAG
GAAGAGGGCCCGGACACTCCCAGCATGGCCTCGGAATTCAACGGGTATCGCTTTC
ACTTGTATGATGTCCAGAAGATGGATCTTTCGATTAGATGACA (SEQ ID NO. 38)

| | | |
|---|---|---|
| NGSAI_NEOTX_39 | MFSD12 | ZRANB3 |

GTAATCCCAGCACTTTGGGAGGCCCAGGTTGGTGGATCACCTGAGGTCAGGAGTT
CGAGACCAGCCTGGCCAGCATGGTGAAACCCCATCTCTACTAAAAATACGAAAAT
TAAGCCAGGCATGGTGTGGGGGGGGGGGGCACCTGTAATCCTCAGCCTCCCCAGT
AGCTGGGACTACAGACGCGTGCCACACCACCTGGCTAATTTTTTGTATTTTTAGTA
GAGATGGGGTTTCACTATGGTGGCCAGGCTGGTCTCAAACTCCTGAGCTCAGGC
(SEQ ID NO. 39)

| | | |
|---|---|---|
| NGSAI_NEOTX_40 | LAMA3 | LOC105372085 |

GTTTCTTCATATGGTGGTTACCTCACTTACCAAGCCAAGTCCTTTGGCTTGCCTGGC
GACATGGTTCTTCTGGAAAAGAAGCCGGATGTACAGCTCACTCTAGATCCACATCT
GTAAATGTCTAAGTCATGCTGCCAGCCAGTCTTGCCTACAGCTACTTGATTCTGGG
AGAGCCTTCTATAAAACTGATTACAGCATTTCCCTGCCACACAGTGAAAAAACAA
TGTAGTTTGATATGATAAAACATTGATT (SEQ ID NO. 40)

| | | |
|---|---|---|
| NGSAI_NEOTX_41 | PDIA4 | UBE2H |

GGGGGGCAAGTGGGGGCTTAGAGGGTGGTAGTGTGGAACACAGTTTAAAAGTCCT
GTCTCCTGTTTCTCTCCCTCCTCCCCATCCCCCCACCGTTTCCCCCTGTTGCAGGGT
TTTGTTTATATAACTCAAGTTGTTTGGCTAAATTCTTCAGATTCTTCTAACAGAGAA
AATGCCATTGAGGATGAAGAGGAGGAGGAGGAGGAAGATGATGATGAGGAAGAA
GACGACTTGGAAGTTAAGGAAGAAAATGGAGTCTTGGTCCTAAATGATGCAAACT
TTGATAATTTTGTGGCTGACAAA (SEQ ID NO. 41)

| | | |
|---|---|---|
| NGSAI_NEOTX_42 | MRPS18A | NA |

AAGGATATTGAGAAAAAATTACGAGGGTAGGTTTTTGAAGATGGCGGCCCTCAAG
GCTCTGGTGTCCGGCTGTGGGCGGCTTCTCCGTGGGCTACTAGCGGGCCCGGCAGC
GACCAGCTGGTCTCGGCTTCCAGCTCGCGGGTTCAGGGAAGCCTGCCGAGTGCCT
GCGATTGCAGGCACGCGCCGCCACGCCTGACTGGTTTTGGTGGAGACGGGGTTTC
GCTGTGTTGGCCGGGCGGTCTCCAGCCCCTAACCGCGAGTGATCCGCCAGCCTTGG
CCTCC (SEQ ID NO. 42)

| | | |
|---|---|---|
| NGSAI_NEOTX_43 | ERP44 | TEX10 |

TGCTGCAGAGCCTGCGGGTGAACAGAGTTGGGCCTGAGGAGCTGCCTGTTGTGGG
CCAGCTGCTTCGACTGCTGCTTCAGCATGCACCCCTCAGGACTCATATGTTGACCA
ATGCGATCTTGGTGCAGCAGATCATCAAGAATATCACGGTAACTTGGGTTTTTACT
CCTGTAACAACTGAAATAACAAGTCTTGATACAGAGAATATAGATGAAATTTTAA
ACAATGCTGATGTTGCTTTAGTAAATTTTTATGCTGACTGGTGTCGTTTCAGTCAGA
TGTTGCATCC (SEQ ID NO. 43)

| | | |
|---|---|---|
| NGSAI_NEOTX_44 | LOC101060341 | LOC284600 |

GTGGATTCCAGAGGGGTGACAGCGAAACGTGGGACCATCCAGTTGCAGGAAAAC
AAGCTTAACACGCCCACTGATTCTACATTATGGCACAGTTCACAGAGGCAGCTGCT
TTGGGAAGTTTGGTGCCAGACCCCGCCAAGCCCCTGCCCGGGGCATCTCCTCCCGC
ACCCTTCGCCGCCATCTTTCAGACGGCTGCTCTCCTGAGCCAGGCCCGCGCGCCAT
CTCCTTTAGGCTCCT (SEQ ID NO. 44)

| | | |
|---|---|---|
| NGSAI_NEOTX_45 | GIPR | IMPAD1 |

AATTTTTGTATTTTTAGTAGAGACGGGGCTTCACTATGTTGGTCAGGCTGGTCTTG
AACTCCTGACCTTGTGTCCTGCCTTCCTCGTCCTCCCAAAGTGCTTGGATTACAGG
CATGAGCCACTGTGCCTGGCCCCTCTTATTTTATTTTTTCGAGACAGAGTTTCACTC
TCGTTGGCCAGGCTGGAGTGCAATGGCGTGATCTCGGCTCACCGCAACCTCTGCTT
CCCGGGTTCAAGCGATTCTCCTGCCTCAGCCTCCCAAGTAG (SEQ ID NO. 45)

| | | |
|---|---|---|
| NGSAI_NEOTX_46 | TMEM241 | WDPCP |

GCTGGGTAGAGATCAACAGCAGTTCAAGATCTCATGTTCTTGTGTGGCTTCCTGCT
TCAGTGCTGTTTGTGGGTATAATCTATGCTGGGTCCAGAGCATTGTCCAGACTGAA
CTCCTGGGACCCTTGGACAGAGGGGATATGCTAAATGAAGCATTTATTGGCCTGTC
TTTAGCACCTCAAGGAGAAGACTCATTTCCAGATAACCTCCCTCCCTCTTGCCCAA
CCCACAGACATATTTTACAACAAAGAATACTGAATG (SEQ ID NO. 46)

| | | |
|---|---|---|
| NGSAI_NEOTX_47 | MUC20 | NA |

CACAGGTCTCTTTCCTCTGTCTTCCTCCATCAGGCTCCGGAAAGCTTTCCCCAGAG
AAGACGCCAGACAGCAGGGGCTGCCTCCCGGGGCTTTTGTGACCCAGCCTGTTTCT
CCATCCGAGCTGCAACCTCTGGGTGGGGGTGTCTGCACCTGCTGCATCAGCCTTTC
TGCCACTCTGGGGTCAGTGAGGTCTTCCGGGCAAGCCACACTCAGCCGCAGGAGG
AGGAAACCTCCATTTTCACCTGCACTCACGTCTGTGGTCGGCCTCGTCCGGGCAGT
CGTGGGCGTGGCTGTTGGGGGC (SEQ ID NO. 47)

| | | |
|---|---|---|
| NGSAI_NEOTX_48 | NA | USP8 |

CTGCAGTGGACTGGGAGGCATGCCAACATGTGCTGGCATCCAAATAACATCCGCC
TCGTATATGGGTCACAGCTGAGCACGTGTTTCATGTCGTGAGTGGGCACTCCAACA
TCGCCTTGAGATTTCATCCTTTTTAAAGTAGCAGCAAGACTTTCTCCATGCAAAAA
GCAGTGCACTGACTGGGCGTGGTGCCTCACAGCTGTAATCCCAACACTCTGGGAG
ACTGAGGTGGGAGGACTGCTTGAGCCCAGGAGTTCAAGAACAGATATTTATGTTG
AGT (SEQ ID NO. 48)

| | | |
|---|---|---|
| NGSAI_NEOTX_51 | NA | VPS45 |

AAAAAACTCAGTATCACTGATCATTAGAGAAATGCAAATCAAAACTATGGTGAGA
TACCATCTCAACACCAGTCAGAATGGCTATTACTAAAAAGTCAAAAAATAATAGA
TGCTGACAAGGTTGTGGAGAAAAGTGAACACTTATTCACCGTTGGTGGGAGTGTA
AATTAGTTCAACCATTGTGGAAGACAGTGTGGCAATTCATCAAAGACCTAAAGGC
AGAAATAGCATTCAACTCAGCAATCCCATTACTGGGTATATACACAACAGAATAT
AAATCATTCTATTATAAAAAGA (SEQ ID NO. 49)

| | | |
|---|---|---|
| NGSAI_NEOTX_52 | LOC107987295 | NRIP1 |

TGGGCTCACTCATGCATCTGCTATCAGCTGGCTGGTTAACTGTAGTTAGTTTATCTT
GATGGCATCATTGGGGAAACTCAGCTCTCTTTCACTGGACTTCTCTTATATTTCTCC
AGCAAACTGGAAAGGGTGTGTTCTCGTGGCAGGGGCAGGAGTCCCAGGCCGCCGC
GGCTCCCAGCCTCCGGCTCCGTCAGGCTCGGTCCGCGAAGGCGCCTGCCGCCCCGT
CCTGGCCCGGCGCCCCGGCGAGCTCTTCCCTCCGACCAGCGGCGCTCACGGCGCA
GCGGCGGAC (SEQ ID NO. 50)

| | | |
|---|---|---|
| NGSAI_NEOTX_53 | NA | TUBB2A |

CTCTAGGCCACCTCCTCCTCAGCCTCCTCCTCGAACTCGCCCTCCTCCTCGGCTGTG
GCATCCTGGTACTGCTGGTACTCGGACACCAGGTCATTCATGTTGCTCTCGGCCTC
GGTGAACTCCATCTCGTCCATGCCCTCGCCCGTGTACCAGTGCAGGAAGGCCTTGC
GCCGGAACATGGCCGTGAACTGCTCGGAGATGCGCTTGAACAGCTCCTGGATGGC
CGTGCTGTTGCCGATGAAGGTGGCCGACATCTTCAGGCCGCGGGGGGGGATGTCG
CACACGGCCGTCTTCACGTTGT (SEQ ID NO. 51)

| | | |
|---|---|---|
| NGSAI_NEOTX_54 | LOC107987295 | NRIP1 |

CCGTGAGCGCCGCTGGTCGGAGGGAAGAGCTCGCCGGGGCGCCGGGCCAGGACG
GGGCGGCAGGCGCCTTCGCGGACCGAGCCTGACGGAGCCGGAGGCTGGGAGCCG
CGGCGGCCTGGGACTCCTGCCCCTGCCACGAGAACACACCCTTTCCAGTTTGCTGG
AGAAATATAAGAGAAGTCCAGTGAAAGAGAGCTGAGTTTCCCCAATGATGCCATC
AAGATGAACTAACTACAGTTAACCAGCCAGCTGATAGCAGATGCATGAGTG (SEQ
ID NO. 52)

| | | |
|---|---|---|
| NGSAI_NEOTX_55 | LOC105379251 | NA |

GCTTAACATAACAATTTTTATTTTTATTACTTCATGTAAGAACTTCTCTACAACCAC
TGATTTTCTTACTTGCTTTCTAAGCAATGTAGAATTTTCGTCACCACTTCACCATTA
ATTTCTTGTTATTAATCCATTGTCGTTTTCCCAGCTCCAGCCTGTTAGATGAGCTCC
TGTCAACCCCAGAGTTTCAGCAAAAGGCACAACCTTTGCTAGATCCGGCGCCACT
GGGGGAGCTGAA (SEQ ID NO. 53)

| | | |
|---|---|---|
| NGSAI_NEOTX_56 | WWOX | WWOX |

CAAAGGCTGCAATCACCTCAAGGCTTAACTAGGGCTGCAGAACCAACTTCGAACG
TGGTTCACTCACATGGCTGTTGGCAGGAGGCTCAGTTCTTCTACACGGGTATGCTT
GAGTATCCTCCCAACATGGCAGCTGGCTTTTCCAGCTGAGGTAGGAGAGGCTGAG
GCAGGAGAATCACTTGATCCCAGGAGGCGGAGGCTGCGGTGAGTTGAGATCACGC
CACTGCACTTCAGCCTGGGTGACAGAGCAAGACTCCATCATGGACTTGGTGAAAG
GCCTCGCCAAGGTAAACAGCAGTGT (SEQ ID NO. 54)

| | | |
|---|---|---|
| NGSAI_NEOTX_57 | NA | URI1 |

TTCCAAATAGACTTTCCTTCCTCGAAACAAATCCAGAGCATCAGCAAAAGGGATCT
TATAAATGGACTTGAACCCCAACTTAAGTCCACTTAAACTTGGTGATGAGGCAAC
AATCTCCTGTTCTCGAAGAGTCTTCTCTTCATCACTTATGTTCTTTCCGGTGCTCAA
CTAAACCTACAGCCTGCTTTGCTGAGCACTTTGCAAACCAGTTGTCCCCCAGTAAA
ACAGTGACTTCATTAGTATGGACAAGTTTTCCTGGCATGAAGGCAAAAGGGC (SEQ
ID NO. 55)

| | | |
|---|---|---|
| NGSAI_NEOTX_58 | CMSS1 | HP09053 |

CTGGCTTTGAGACAACGTGATTCTCCGCAGCTGGTCGCCTACCCGTGATGTTCTGC
CCACGTCGAGACCTGAGCTGAAATGGCAGACGATCTCGGAGACGAGTGGTGGGAG
AACCAGCCGACTGGAGCAGGCAGCAGCCCAGAAGCATCAGATGGTGAAGGAGAA
GGAGACACAGAAGTGATGCAGCAGGAGACAGTTCCAGTTCCTGTACCTTCAGAGA
AAACCAAACAGCCTAAAGAATGTTTTTTGATACAAC (SEQ ID NO. 56)

| | | |
|---|---|---|
| NGSAI_NEOTX_59 | CRLS1 | NA |

TGGGACTACAGGCGTGTGCCACCACACCTGCCTAATTTTTTGCATTTTTTTTTTTTT
AGTAGAGACGGGGTTTCACCATGTTAGCCAGGATGGTCTTGATCTGACCTCGTGAT
CCACCCGCCTCAGCCTCTCAAAGTGCTGGGATTACAGGTGTGAGCCACTGTGCCCA
GCCACTAATTTTTTGTATTATTATTTTTTGTAGAAACAGGGTCTCACTATGTTGCCC
AGGCTGG (SEQ ID NO. 57)

| | | |
|---|---|---|
| NGSAI_NEOTX_60 | FGF12 | NA |

CACTACACGCAGGCCCACGGGAATTAGATTGAAGAGAGTGTAGTCGCTGTTTTCG
TCCTTGGTCCCATCAATGGTACCATCTGGGTGCATCTGCAGGAAGTATCCCTGCTG
GCTGAATAACCTTGTCACAATCCCTTTGAGCTGGGGTTCTTTGCTCTCCATTTCGGT
CCCTTTCGAGTGCTGGGAAGTTCAATGGAAGTTGGCCGGAAGATGTGGGCCCGCT
TCAGATTCCCAAATCTGGGAAGCCAATCTGATGATTTCGCCCGTACTTCCTTCCTTC
CCCTCAGGCTTCCTTTTTTTT (SEQ ID NO. 58)

| | | |
|---|---|---|
| NGSAI_NEOTX_61 | ADAP1 | SUN1 |

ACGCCGCGAGAGCCAGGTTTGAGTCCAAAGTACCCTCCTTCTACTACCGGCCCAC
GCCCTCCGACTGCCAGCTCCTTCGAGAGCAGTGGATCCGGGCCAAGTACGAGCGA
CAGGAGTTCATCTACCCGGAGAAGCAGGAGCCCTACTCGGCAGCCTGACATTTAC
CCCGGTAACTGCTGGGCATTTAAAGGCTCCCAGGGGTACCTGGTGGTGAGGCTCTC
CATGATGATCCACCCAGCCGCCTTCACTCTGGAGCACATCCC (SEQ ID NO. 59)

| | | |
|---|---|---|
| NGSAI_NEOTX_64 | CMSS1 | HP09053 |

CCTGGTCTTGGTGGTATTCTCTTTTCTTTCCTTTGGTTGTATCAAAAAACATTCTTTA
GGCTGTTTGGTTTTCTCTGAAGGTACAGGAACTGGAACTGTCTCCTGCTGCATCAC
TTCTGTGTCTCCTTCTCCTTCACCATCTGATGCTTCTGGGCTGCTGCCTGCTCCAGT
CGGCTGGTTCTCCCACCACTCGTCTCCGAGATCGTCTGCCATTTCAGCTCAGGTCT
CGACGTGGGCAGAACATCACGGGTAGGCGACCAGCTGCGGAGAATCACGTTGTCT
CAAAGCCAGGCGGCCGGCG (SEQ ID NO. 60)

| | | |
|---|---|---|
| NGSAI_NEOTX_65 | LOC105371307 | LOC105371308 |

CCAAATCTTATTGGATGGTTGGTATGTATCAAGGATTGTTTTACCCTCATTTAATCT
TCTCAGTAATTCAATGATTTGGAACGCTTAAAGCATTCAAAAGAATAAAATTATAG
CTTCTGCAGCAACATGGATGGAACTGGAGGCCATAATCAGGTTTGAAAATGGCTT
GTGATTCTTCCTCCATTTCAGTGTCCAACAAGCTCAGTTAGAACGTAAATGCAAGT
CCTACAGCATTCAGAGGTTCCCAAACTTTCTCAGTTTTAATGCCCTTTGTCAGAAA
TCTCTTGGTGCCCCAGCAACC (SEQ ID NO. 61)

| | | |
|---|---|---|
| NGSAI_NEOTX_66 | DNAAF5 | NA |

GGGAGCCCTGAGCTTGTTTTCCTGCAACTAGACGGTCCCATGTGGGGACGATGGG
AGACAGTGACGGATCATCAGGCATTAGTTTCATAAGGAGCGTCAGCTTGGATCCC
TCGCGTGCACAGTTCACAATAGGATTTGTGCTCCTATGAGAATCTAATGCCGTTGC
CGATCTGACAGGAGGCAGAGCTCAGGTGGTAATGCTCGTTTGCCTGCCACTCACCT
CCTGCTGTGTGGCCTGGTTCCTAACAGGTCA (SEQ ID NO. 62)

| | | |
|---|---|---|
| NGSAI_NEOTX_67 | LOC105371662 | LOC105371664 |

ACTTTTATAAGCTCGACTCACATGACGAAAGCCCTCATCAGATGCTTACATCATGA
TCTTGGACTTCCCAGCCTCCAGACTGATGCTATGGAAGATCAGAAAATATAAATTT
ATGAACTGCTATAAACTGTTATTTTCTTCGTGAAGATCAGACATGTGGCAGGCAAG
TTAATCTTCAGTGGAATATGCAAATAGGATTTCTGAATTTGGCATGCAAATGAATT
TGAGAGCTTCTGGGAGCATCTCTTCCAAGATTCTGGTAAGCCTTTCTTCCTGGGCG
AAACTTAGCAGAGGAAGGTAT (SEQ ID NO. 63)

| | | |
|---|---|---|
| NGSAI_NEOTX_68 | NA | PTGR1 |

GGGAAGCGAGGAGCGCCTCTTCCCCGCCGCCATCCCATCTAGGAAGTGAGGAGCG
TCTCTGCCCGGCCGCCCATCGTCTGAGATGTGGGGAGCACCTCTGCCCCGCCGCCC
TGTCTGGGATGTGAGGAGCGCCTCTGCTGGGCCGCAACCCTGTCTGGGAGGTGAG
GAGCGTCTCTGCCCGGCCGCCCCGTCTGAGAAGTGAGGAAACCCTCTGCCTGGCA
ACCGCCCCGTCTGAGAAGTGAGGAGCCCCTCCGTCCGGCAGCCACCCCGTCTGGG
AAGTAGGTGGAGAGTTTTCAAACAC (SEQ ID NO. 64)

| | | |
|---|---|---|
| NGSAI_NEOTX_69 | B4GALT5 | NA |

AAAAAACACAAAAATTAGCCGGGCATGGTGGCAGGTACTTGTAATCTCAGCTACT
CAGGAGGCTGAGGAAGGAGAATCGCTTGAACCCAGGAGGCAGAGGTTACAGTGA
GCTGAGATCACACGGTTGCACTCCAGCCTGGGCAACAACAGCAAAACTCCATTTC
AAAAAAACAAAGTGGCCACTGGACCAGGCACAGTGGCTCGCGCCTGTAATCCCAG
CACTTTGGGAGGTTAAGGCAGGTGGATCACCTGAAGTCAGGAGTTCGAG (SEQ ID
NO. 65)

NGSAI-NEOTX-ID fusions can be characterized by their sequence around the fusion point, by their fusion partners (e.g., gene name) if any, and by the score describing the predicted accuracy of the fusion.

Additional derived features of said fusions are their
expression estimates, as a marker of epigenetic changes in the tumor;
locations at single or multiple loci, using a chromosome coordinate reference genome;
coding capability;
exon capabilities (and splicing events);
transmembrane containing domain;
other protein domain detections;
expression as non-coding RNAs (e.g., lnc-, mi-, sno-, or piRNAs)

In some embodiments of the invention, the at least one gene fusion, non-gene fusion, genomic alteration, transcriptomic alteration or neotranscript is a fusion in a single gene/non-gene. The at least one gene fusion, non-gene fusion, genomic alteration, transcriptomic alteration or neotranscript is a fusion of a multiple chromosomal loci. For example, fusions contemplated and disclosed herein may comprise at least 2, 3, 4, 5, 6, or more distinct chromosomal loci. Such loci may correspond to such loci may comprise coding or non-coding regions. Similarly, such loci may comprise genes or regions between genes. In some preferred embodiments, the at least one gene fusion, non-gene fusion, genomic alteration, transcriptomic alteration or neotranscript is a fusion of at least 2 distinct chromosomal loci. Alternatively, the at least one gene fusion, non-gene fusion, genomic alteration, transcriptomic alteration or neotranscript is a fusion of at least 3 distinct chromosomal loci. In further embodiments, the at least one gene fusion, non-gene fusion, genomic alteration, transcriptomic alteration or neotranscript is a fusion of at least 4 distinct chromosomal loci.

In some embodiments, the at least one gene fusion, non-gene fusion, genomic alteration, transcriptomic alteration or neotranscript comprises or is transcribed from at least one of the genes set forth in Table 1. In some such embodiments, the at least one gene fusion, non-gene fusion, genomic alteration, transcriptomic alteration or neotranscript comprises or is transcribed from at least one sequence at least 80% homologous to at least one of the provided genes set forth in Table 1. Preferably, the gene fusion, non-gene fusion, genomic alteration, transcriptomic alteration or neotranscript comprises or is transcribed from at least one sequence selected from SEQ ID Nos. 1-47. In some such embodiments, said gene fusion, non-gene fusion, genomic alteration, transcriptomic alteration or neotranscript comprises or is transcribed from at least one sequence at least 80% homologous to a gene of SEQ ID Nos. 1-47.

In some embodiments, the gene fusions or non-gene fusions disclosed herein are transcribed in a cancer cell, resulting in transcriptomic alteration and/or the synthesis of at least one neotranscript. The fusions disclosed herein, may be intrachromosomal (e.g., fusions arising from rearrangements occurring within a chromosome as are known in the art, such as duplications/amplifications, insertions, deletions, inversions, and the like) or interchromosomal (e.g., fusions arising from rearrangements occurring between two or more chromosomes, such as translocations or more complex structural genome variations as are known in the art, including but not limited to complex chromosomal rearrangements such as insertion-translocations, inversions associated with copy number variation, translocations affecting more than 2 chromosomes, and combinations thereof).

In some embodiments the sample is a liquid or tissue biopsy.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See for example and without limitation, “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000). Similarly, chemistry terms used herein, unless otherwise defined herein, are used according to conventional usage in the art.

All of the above, and any other publications, patents and published patent applications referred to in this application are specifically incorporated by reference herein.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The cancer of the disclosed invention can be any cell in a subject undergoing unregulated growth, invasion, or metastasis. Cancer, as disclosed herein, includes both solid and liquid tumors including, for example, brain cancers including glioblastoma, tenosynovial giant cell tumors (TSGCTs), sarcoma, melanoma, mesothelioma, uterine cancer, prostate cancer, kidney cancer, gall bladder cancer, cervical cancer, bladder cancer, ovarian cancer, lung cancers, adenocarcinoma of the lung, thyroid cancer, bladder cancer, breast cancer, esophageal cancer, endometrial cancer, gastric cancer, gastrointestinal cancer, renal cancer, adrenal cancer, mullerian cancer, Merkel carcinoma, acute lymphoblastic cancer, colorectal cancer, pancreatic cancer, liver cancers including hepatocellular carcinoma, AML, DLBCL, lymphomas, multiple myelomas, and the like. In some embodiments, the cancer is a gallbladder cancer, exocrine adenocarcinoma, or apocrine adenocarcinomas. Preferably the cancer is breast cancer, prostate cancer, or pancreatic cancer. Most preferably, pancreatic cancer.

In some embodiments, the cancer can be any neoplasm or tumor for which radiotherapy or chemotherapy is currently used. Alternatively, the cancer can be a neoplasm or tumor that is not sufficiently sensitive to radiotherapy or chemotherapy using standard methods. Thus, the cancer can be a sarcoma, lymphoma, leukemia, carcinoma, blastoma, or germ cell tumor. A representative but non-limiting list of cancers of the disclosed invention include hepatocellular carcinoma, lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, renal cancer, prostatic cancer, and pancreatic cancer.

Also provided herein are methods comprising performing a bioassay to detect at least one gene fusion, non-gene fusion, genomic alteration, transcriptomic alteration, or neotranscript comprising or transcribed from at least one of the genes set forth in Table 1 in a sample from a subject, receiving the results of the bioassay into a computer system, processing the results to determine an output, presenting the output on a readable medium, wherein the output identifies therapeutic options recommended for the subject based on the presence or absence of the at least one gene fusion, non-gene fusion, genomic alteration, transcriptomic alteration, or neotranscript, wherein the sample is a liquid or tissue biopsy. In some embodiments, the at least one gene fusion, non-gene fusion, genomic alteration, transcriptomic alteration, or neotranscript comprises or is transcribed from at least one sequence at least 80% homologous to at least one of the genes set forth in Table 1. The at least one gene fusion, non-gene fusion, genomic alteration, transcriptomic alteration, or neotranscript may be a fusion of at least 2, 3, 4, 5, or 6 distinct chromosomal loci as described herein. In some embodiments, the at least one gene fusion, non-gene fusion, genomic alteration, transcriptomic alteration or neotranscript is a fusion of at least 2 distinct chromosomal loci. The at least one gene fusion, non-gene fusion, genomic alteration, transcriptomic alteration or neotranscript may be a fusion of at least 3 distinct chromosomal loci. In other embodiments, the at least one gene fusion, non-gene fusion, genomic alteration, transcriptomic alteration or neotranscript is a fusion of at least 4 distinct chromosomal loci. In preferred embodiments, the bioassay comprises probes specific for a fusion locus comprising a sequence set forth in Table 1.

In some aspects of the invention, provided herein are cancer diagnostic kits comprising at least one reagent allowing the detection of at least one gene fusion or non-gene fusion in a sample from a subject, wherein said fusion comprises or is transcribed from at least one of the genes set forth in Table 1. In some embodiments, the fusion comprises a DNA sequence at least 80% homologous to at least one of the genes set forth in Table 1. In other embodiments, the fusion comprises or is transcribed from at least one sequence set forth in Table 3. The fusion may comprise or be transcribed from at least one sequence with at least 80% homologous to a gene set forth in Table 3. In some embodiments, the fusion is transcribed in a cancer cell, resulting in the synthesis of at least one transcriptomic alteration, or neotranscript. In some embodiments, the fusion is intra or interchromosomal. In some such embodiments, said fusion arises from chromosomal rearrangements as disclosed herein.

In some embodiments, the kit comprises a set of probes, wherein each probe specifically hybridizes to a nucleic acid comprising the sequence set forth in set forth in Table 1 or Table 3. In some such embodiments, the probes are capable of hybridizing or otherwise binding to the fusion locus (e.g., a locus comprising the sequence set forth in Table 1 or Table 3. Preferably, such probes comprise: a nucleic acid sequence configured to specifically hybridize to the nucleic acid comprising the fusion locus, and a detectable moiety covalently bonded to the nucleic acid sequence. In preferred embodiments, the fusion locus comprises at least one sequence set forth in Table 1 or Table 3. In some embodiments, the sample is a liquid or tissue biopsy. In some embodiments, the cancer is selected from: pancreatic cancer, Merkel carcinoma, Acute Myeloid Leukemia, Metastatic Carcinoma, prostate cancer, adrenal cancer, mullerian cancer, uterine cancer, kidney cancer, gall bladder cancer, cervical cancer, bladder cancer, ovarian cancer, breast cancer, head and neck cancer, esophageal cancer, lung cancer, liver cancer, colon cancer, gastrointestinal cancer, colorectal cancer, Acute lymphoblastic cancer, lymphoma, sarcoma, melanoma and brain cancer.

In certain aspects, provided herein are compositions comprising at least one of the following: (a) a detection probe comprising an oligonucleotide sequence that hybridizes to a junction of a gene fusion, non-gene fusion, genomic alteration, transcriptomic alteration or neotranscript comprising at least one sequence selected from SEQ ID Nos. 1-65; (b) a first labeled probe comprising an oligonucleotide sequence that hybridizes to a 5′ portion of a gene fusion, non-gene fusion, genomic alteration, transcriptomic alteration, or neotranscript comprising or transcribed from at least one sequence selected from SEQ ID Nos. 1-65, and a second labeled probe comprising an oligonucleotide sequence that hybridizes to the corresponding 3′ portion of the gene fusion, non-gene fusion, genomic alteration, transcriptomic alteration, or neotranscript; (c) a first amplification oligonucleotide comprising a sequence that hybridizes to a 5′ portion of a gene fusion, non-gene fusion, genomic alteration, transcriptomic alteration, or neotranscript comprising or transcribed from at least one sequence selected from SEQ ID Nos. 1-65, and a second amplification oligonucleotide comprising a sequence that hybridizes to the corresponding 3′ portion of the gene fusion, non-gene fusion, genomic alteration, transcriptomic alteration, or neotranscript; (d) an antibody that specifically binds to an amino acid sequence encoded by at least one sequence selected from SEQ ID Nos. 1-65 and (e) an in situ hybridization probe for detecting a gene fusion, non-gene fusion, genomic alteration, transcriptomic alteration or neotranscript comprising at least one sequence selected from SEQ ID Nos. 1-65. In some embodiments, the gene fusion, non-gene fusion, genomic alteration, transcriptomic alteration or neotranscript is derived from a sample comprising a prostate cell or fraction, a prostatic secretion or fraction, or a combination thereof. In other embodiments, the gene fusion, non-gene fusion, genomic alteration, transcriptomic alteration or neotranscript is derived from a sample comprising a breast cell or fraction, a breast secretion or fraction, or a combination thereof. In further embodiments, the gene fusion, non-gene fusion, genomic alteration, transcriptomic alteration or neotranscript is derived from a sample comprising a pancreatic cell or fraction, a pancreatic secretion or fraction, or a combination thereof. In preferred embodiments, the sample is a liquid or tissue biopsy.

In some embodiments the detection probes, labeled probes, in situ hybridization probes, or amplification oligonucleotides of the invention do not hybridize under stringent hybridizing conditions to DNA or RNA that is not part of, or results from, the gene fusion, non-gene fusion, genomic alteration, transcriptomic alteration, or neotranscript.

In some embodiments, the first and second amplification oligonucleotides do not amplify DNA or RNA that is not part of, or results from, the gene fusion, non-gene fusion, genomic alteration, transcriptomic alteration, or neotranscript. Also provided herein are kits and packaged assays comprising the compositions of the invention.

### EXAMPLES

The invention now being generally described, it will be more readily understood by reference to the following examples, which are included merely for purposes of illustration of certain aspects and embodiments of the present invention, and are not intended to limit the invention.

### Example 1: Materials and Methods

### Method and Pipeline to Detect Neotranscripts Fusion

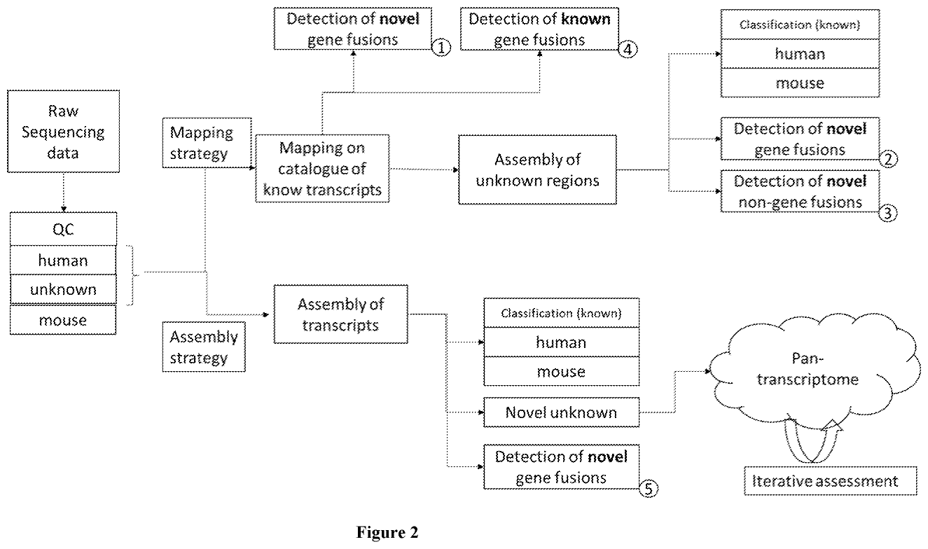

A set of tools were used to create the analytical pipeline described in FIG. 2. These tools were managed by a pipeline reproducible standard (NextFlow®, Seqera Labs, Spain). The pipeline is a set of commands issued to transform the raw data in a usable dataset that is then submitted to several controls and discovery tools. The assemblage of all the software is considered as the discovery tool for the neotranscipts/fusions.

In order to identify data that are of good quality, softwares like FastQC, BBMap, SeqTK, Bedtools, Samtools, PacBio-CCS, Lima, Isoseq3 were used to transform raw sequencing data into usable and more reliable data. These data were then submitted to several softwares that quantify and assess the neotranscripts/fusions, such as Kallisto and Mininmap2. To identify coding capacity of genes, the CD-Hit software was used to assess the completeness and coding potential for all novel neotranscripts/fusions. Finally, data representation, visualization and assessment were made by both R-stat and IGV. These and other software used in such analysis are presented in Table 2.

TABLE 2
Applied Software

| Tools | | |
|---|---|---|
| name | Description | Reference/github |
| Xengsort | This tool, xengsort, uses 3-way bucketed Cuckoo hashing | gitlab.com/genomeinformatics/xengsort/ |
| | to efficiently solve the xenograft sorting problem | |
| FastQC | FastQC aims to provide a simple way to do some quality | Andrews S. (2010). |
| | control checks on raw sequence data coming from high | FastQC: a quality |
| | throughput sequencing pipelines. It provides a modular | control tool for high |
| | set of analyses which you can use to give a quick | throughput sequence |
| | impression of whether your data has any problems of | data. Available online |
| | which you should be aware before doing any further | at: |
| | analysis. | http://www.bioinformatics.babraham.ac.uk/projects/fastqc |
| BB2map | BBTools is a suite of fast, multithreaded bioinformatics | igi.doe.gov/data-and- |
| | tools designed for analysis of DNA and RNA sequence | tools/bbtools/ |
| | data. BBTools can handle common sequencing file | |
| | formats such as fastq, fasta, sam, scarf, fasta + qual, | |
| | compressed or raw, with autodetection of quality | |
| | encoding and interleaving. | |
| Kallisto | kallisto is a program for quantifying abundances of | Nicolas L Bray, Harold |
| | transcripts from bulk and single-cell RNA-Seq data, or | Pimentel, Páll Melsted |
| | more generally of target sequences using high- | and Lior Pachter, Near- |
| | throughput sequencing reads. | optimal probabilistic |
| | | RNA-seq |
| | | quantification, Nature |
| | | Biotechnology 34, 525- |
| | | 527 (2016), |
| | | doi: 10.1038/nbt.3519 |
| Minimap2 | Minimap2 is a versatile sequence alignment program that | github.com/lh3/minimap2 |
| | aligns DNA or mRNA sequences against a large | |
| | reference database. | |
| SPADES | SPAdes - St. Petersburg genome assembler - is an | github.com/ablab/spades |
| | assembly toolkit containing various assembly pipelines. | |
| Samtools | Samtools at GitHub is an umbrella organisation | samtools.github.io |
| | encompassing several groups working on formats and | |
| | tools for next-generation sequencing: | |
| Bedtools | Collectively, the bedtools utilities are a swiss-army knife | bedtools.readthedocs.io |
| | of tools for a wide-range of genomics analysis tasks. | |
| SeqTK | Seqtk is a fast and lightweight tool for processing | github.com/lh3/seqtk |
| | sequences in the FASTA or FASTQ format. | |
| CD-Hit | CD-HIT is a very widely used program for clustering and | Clustering of highly |
| | comparing protein or nucleotide sequences. | homologous sequences |
| | | to reduce the size of |
| | | large protein database″, |
| | | Weizhong Li, Lukasz |
| | | Jaroszewski & Adam |
| | | Godzik Bioinformatics, |
| | | (2001) 17: 282-283 |
| R-stat | R is a free software environment for statistical computing | r-project.org |
| | and graphics. | |
| PacBio - | CCS combines multiple subreads of the same SMRTbell | github.com/PacificBiosciences/ccs |
| CCS | molecule using a statistical model to produce one highly | |
| | accurate consensus sequence, also called a HiFi read, | |
| | along with base quality values. This tool powers the | |
| | Circular Consensus Sequencing workflow in SMRT | |
| | Link. | |
| Lima | Lima, the PacBio barcode demultiplexer, is the standard | github.com/PacificBiosciences/barcoding |
| | tool to identify barcode sequences in PacBio single- | |
| | molecule sequencing data. | |
| Isoseq3 | IsoSeq v3 contains the newest tools to identify transcripts | github.com/PacificBiosciences/IsoSeq |
| | in PacBio single-molecule sequencing data | |
| Nextflow | Nextflow enables scalable and reproducible scientific | nextflow.io |
| | workflows using software containers. I | |
| IGV | The Integrative Genomics Viewer (IGV) is a high- | software.broadinstitute.org/software/igv/ |
| | performance, easy-to-use, interactive tool for the visual | |
| | exploration of genomic data | |

### Example 2: Experimental Design

Collectively, a collection of over 2500 human tumors, termed patient-derived xenografts (PDX), were isolated and propagated in vivo by grafting in mice. A subset of these PDX samples was analyzed by next-generation sequencing of their genomic DNA and transcriptomic RNA, generating a database of the genomic and inferred epigenetic characteristics of over 1500 of those tumors.

The obtained raw sequences were first compared to the human and mouse genomes, in order to remove the murine DNA and RNA that contaminate the human tumors explanted from mice, as illustrated in FIG. 2. The selected human or unknown sequences were then either aligned to previously reported human gene or RNA sequences, or assembled de novo. This yielded a collection of sequences of known fusions as well as unknown human gene or non-gene candidate fusions that may be specific to the analyzed human tumor cells.

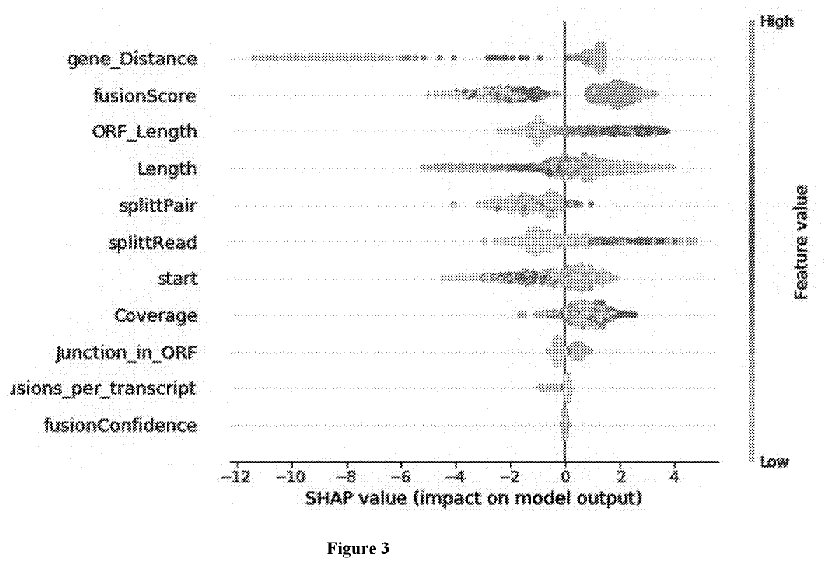

In order to assess the robustness of the fusion selection process and to provide a confidence score for the candidate fusions, a machine learning approach was used to determine which fusion features had a positive (dark grey) or negative (light grey) effect on the predictive value of candidate neotranscript and/or genomic fusion when considering known fusions (FIG. 3). This provided a score representing the likelihood that a given fusion sequence candidate represents a fusion truly occurring in cancer cells rather than a sequencing artifact.

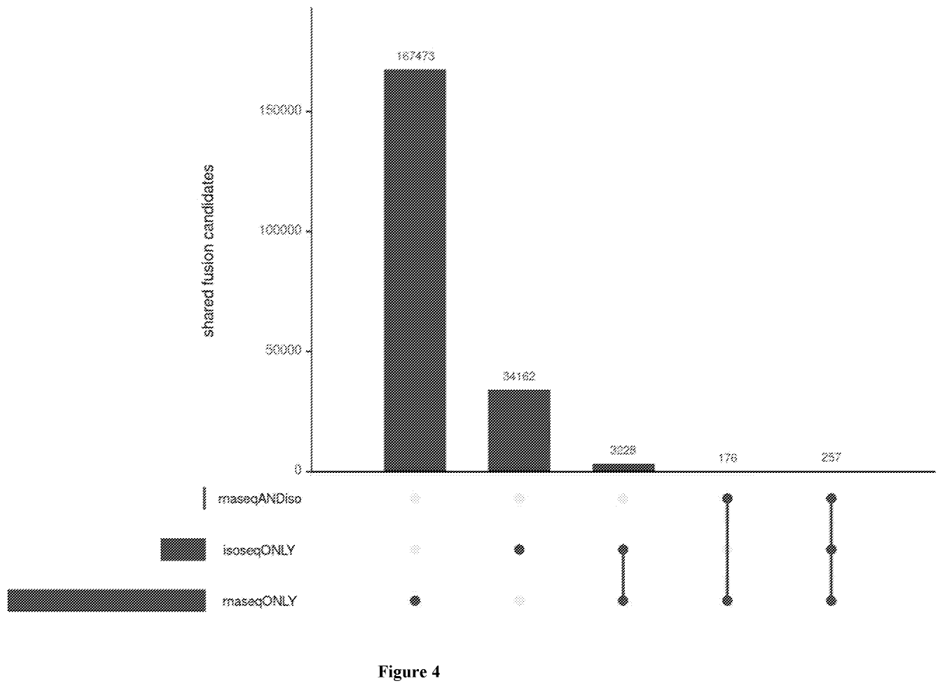

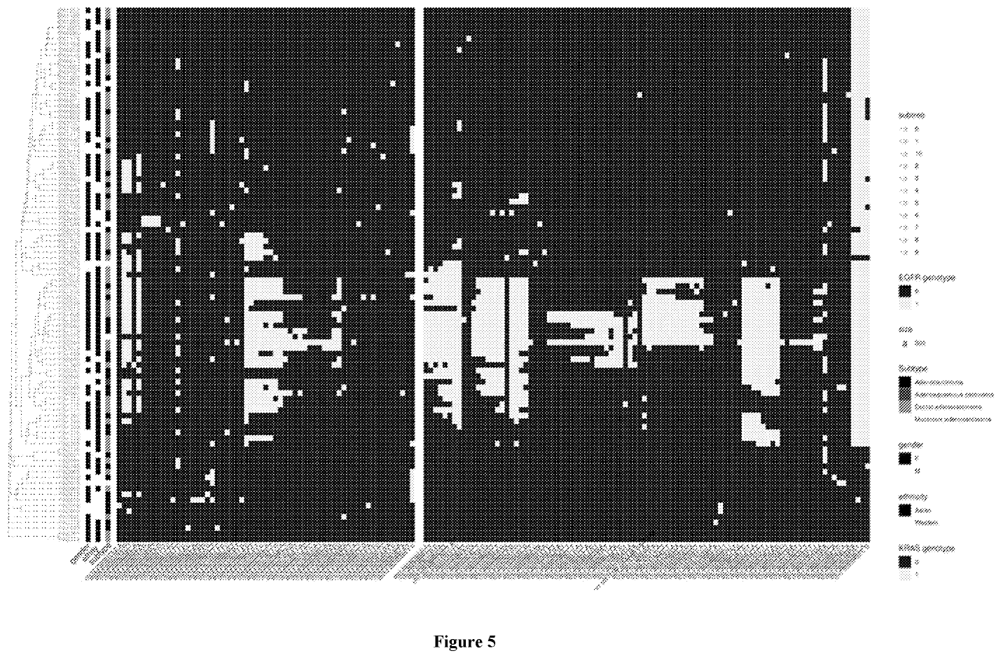

To further exclude fusion artifact sequences that may be linked to a particular DNA sequencing technology, analysis was performed using several sequencing approaches. Two distinct NGS approaches were compared, namely Illumina® RNAseq short RNA reads and PacBio® long genomic DNA and RNA reads obtained from 136 PDX pancreatic cancer models. Use of the sequence datasets obtained from either sequencing strategy yielded 20,811 or 81,466 candidate fusions, respectively (FIG. 4). However, use of combinations of both datasets yielded a total of 433 more reliable candidate fusion sequences.

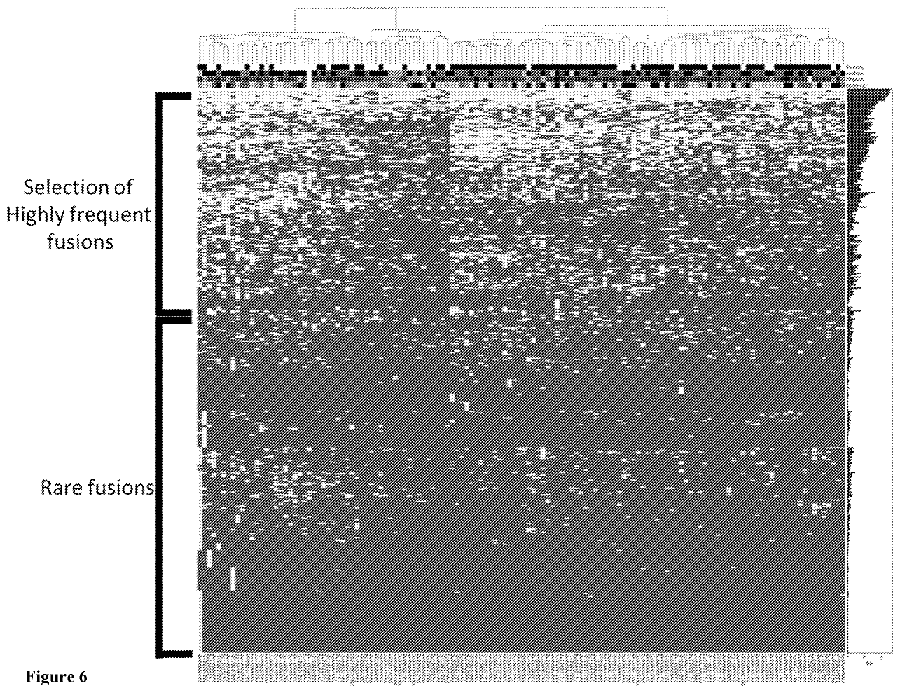

To further validate the selected approaches and datasets of fusion sequences, genomic alterations known to occur in pancreatic cancer biopsies were searched in the 433 fusion sequence dataset. As expected, mutations were found in the epidermal growth factor receptor (EGFR) and Kirsten Ras (KRAS) genes, with a clear over representation of KRAS mutations (FIG. 5). This correlated well with previous reports indicating that over 90% of human pancreatic cancers harbor KRAS alterations, while EGFR and KRAS mutations cross-talk was involved in metastasis formation in the most aggressive cancers types (Fitzgerald et al., 2015), thus providing a further validation of the PDX model and neotranscript/fusion datasets. Having validated the novel cancer-specific neotranscript/fusion dataset, whether it might constitute a basis for the identification of markers specific to various cancer types and subtypes, for IVD use was then evaluated.

### Example 3: Prevalence of Identified Neotranscripts/Genomic Fusions

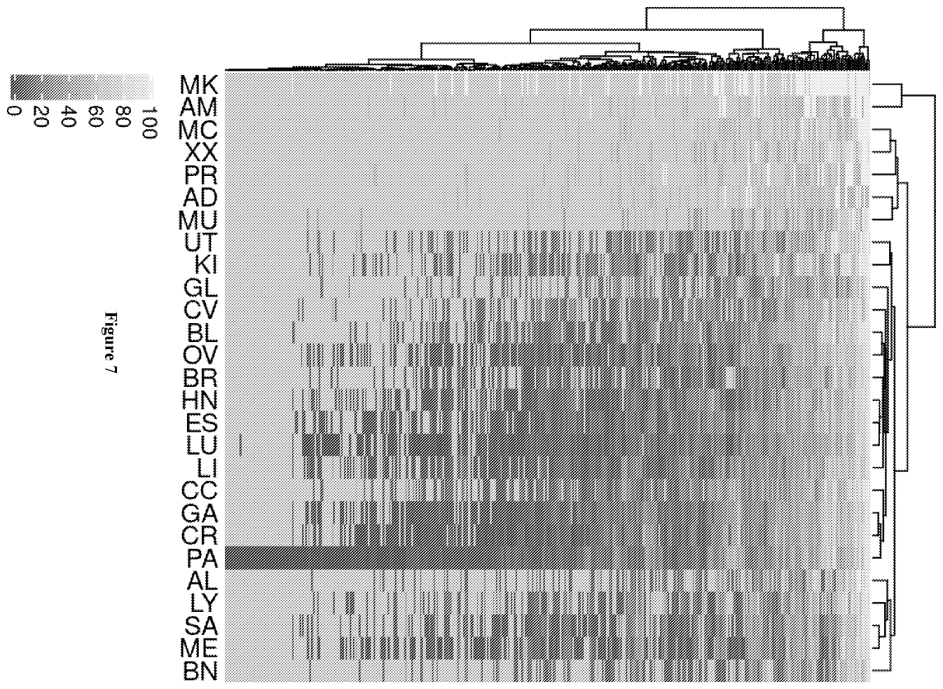

The prevalence of the identified 433 neotranscripts/genomic fusions among the 136 pancreatic cancers samples was assessed, showing some that occur in nearly all pancreatic cancer types, whereas others only occurred in few samples (see top and bottom lines of FIG. 6, respectively). Thus, a specific subset of fusions was present in nearly 100% of all pancreatic cancer samples (see frequency diagram on the right-hand side of FIG. 6).

Some cancer samples were found to harbor higher loads of such fusions than others, which is consistent with the expected heterogeneity among the various subtypes of pancreatic cancers and can be used to describe pancreatic cancer subtypes. The Pancreatic ductal adenocarcinoma (PDAC), a highly aggressive lethal pancreas malignancy that lacks an early diagnostic assay and displays limited response to available treatments (Sarantis et al., 2020), is often diagnosed in Asian patients and displayed a tendency to cluster together. These fusions did not cluster with those observed from pancreatic adenocarcinoma of Western patients, which mostly clustered together and showed a high occurrence of distinct sets of fusions (see left-hand side columns of FIG. 6). Consistently, clusters of samples associated to a poor prognosis were different when considering tumors of different ethnic origins (see the pathology description line under the clustering at the top of FIG. 6). Specific sets of fusions can allow the subtyping of pancreatic cancer subtypes, when taken together with other parameters such as the patient ethnic origin.

### Example 4: Specificity to Pancreatic Cancers

Whether some of these 433 neotranscripts/genomic fusions might be specific to pancreatic cancers was assessed, as similar mutations or chromosomal aberrations often occur in different tumor types. A set of 47 neotranscripts/fusions was observed to occur exclusively in pancreatic cancer and in no other PDX cancer types (FIG. 7). This indicated that the detection of these markers in clinical extracts, such as blood samples, can be taken as an early indication of the onset of a pancreatic cancer, in an IVD or prognostic evaluation of patients.

TABLE 3
Neotranscripts/fusions was observed to occur exclusively in
pancreatic cancer

| | Cancer | | |
|---|---|---|---|
| NGSAI_ID | Specificity | Gene1 | Gene2 |
| NGSAI_NEOTX_1 | Pancreatic | SPPL2A | USP8 |

ATGAGAGTTGGAGAAAAAACTACTGGCTTGGTGGAACGATCAGGAATAACTTTTC
CACTCTGAGGAGATTGTTGCTCATGGTTTGTACTTTCAGATTTTATTCTATGCTCTT
CAGGCAATTTGACTGCTGGTTTTTTAGTACGATCAATCTTGAAGTTGGGCAACTTC
AGTGTTTTAATTAAATTCAGACAGAAAGCAATCCCCAAGATATCCTGTAAAATCCA
AGCCCACCTGTCTTCATTTCGAAACACAGCCCAAACAACAGCTACTGCTATGCACA
GTCCAGAGAGAAAAATAAGTC (SEQ ID NO. 1)

| | | | |
|---|---|---|---|
| NGSAI_NEOTX_2 | Pancreatic | FBRSL1 | LOC105370091 |

GAGGGTACCGAGATTGGCCGTCGGCTGGCAGGCGCCCAGGAGAGCCGGTGGCGT
GAGCTCCAAGCCTGAAGGCAGGGGAGGACCCACGTCCCAGCCCGAATCGAGCAG
TGTGTGTGAACACTCCCTGCCTCGGCCTTTCTGTCCTACTCAGGCCTCGCCGGCGC
CCCAGGCAGTCGCCCCTAGTCCCGGGGCCGGAGCCGGGCTGCATGGACGCGGGCG
TGGAGCGCGAGCCCCGGGTGGCCCTGGCCCGTCCAGGCGACCCCTCTCCCCGCGT
GCCCTGCTCAGCCGGAGCTCGGGCCGG (SEQ ID NO. 2)

| | | | |
|---|---|---|---|
| NGSAI_NEOTX_3 | Pancreatic | MIRLET7BHG | PAK4 |

AAGTTGGACGGCGCGGAGATCTCCACCCGCTTCTTCCTCTTCCCAAACATGGTGCC
GGGGACTCGGTGCGGCCTGCACACACCTGGTCTGATGCTGGTGGGACAGAAGTGC
CCTCAGGCAGGTGACCACTCCTCTAGGGGCTTCGGGTTACTCATCCGAGGTGCCGG
AGGATGGAGGCGTCTTCTCCAAAGCCAGGAAGTGAAAATGACGTCCCTGGGCCCA
GCCGGTCACCCGGGTGGGGGAGGAGGGCAGGTCCCGCCGGCCAGCAGGCTGCCC
GGTGCCAGCCCCAGCTATGGGCCCA (SEQ ID NO. 3)

| | | | |
|---|---|---|---|
| NGSAI_NEOTX_4 | Pancreatic | PAK4 | PRR34-AS1 |

CTCGAAGTTGGACGGCGCGGAGATCTCCACCCGCTTCTTCCTCTTCCCAAACATGG
TGCCGGGGACTCGGTGCGGCCTGGTCTGATGCTGGTGGGACAGAAGTGCCCTCAG
GCAGGTGACCACTCCTCTAGGGGCTTCGGGTTACTCATCTTTTCACGGGAAGTGAC
CCTCCTCCCCATGGGTCAATAAGTTAACGCCAAATCGCGGCAAAACGGCGAATTC
CATCTCTGAGGCTCTAGAAGCTCAATCTTCTGGGCCCCTGGCTCCTGGCCTCGGGT
CCTGCTGGTGCCCAGGTCGCCCG (SEQ ID NO. 4)

| | | | |
|---|---|---|---|
| NGSAI_NEOTX_5 | Pancreatic | LOC400682 | ZNF85 |

AGCATTGACAATAGGCACAATATAGTTTTGCATTGGTGTCTGTGAATTTGATAGAG
CAAACACTTCTTCAAGTTGTTTTTTTTGTTTTGTTTTGTTTTTTTGTTTTTTTTTTGAG
ACAGCATTTTGCTCTTGTTGCCCAAGCTGGAGTGCAGTGTCATGATCTTGGCTCAC
AGCAACCTCCGCCTCCCGGGTTCAAGTGATGCTCATTTCTAGGCTTCCAGGAGGAC
CTGGCGTCTTAGCTGGGGATCTCCCAATACCTGCAGGTCACAGGGCCACAGAGGC
TGGGCCCCTAGGAGAAGAG (SEQ ID NO. 5)

| | | | |
|---|---|---|---|
| NGSAI_NEOTX_6 | Pancreatic | DOCK1 | FAM196A |

GGCCTGCTGTCAGCTGCTCAGCCACATCCTGGAGGTGCTGTACAGGAAGGACGTG
GGGCCAACCCAGAGGCACGTCCAGATTATCATGGAGAAACTTCTCCGGACCGTGA
ACCGAACCGTCATTTCCATGGGACGAGATTCTGAACTCATTGCTTTCATATCGGAC
GGGGCAAGACACAGCTAATTGTGACACATGCAGGAACAGTGCATGTATTATCTAT
AGTGTGGAGCTGGATTTTAAGCAGCAAGAAGACAAACTCCAGCCGGTTCTAAGAA
AACTCCACCCTATTGAGGAAACTCA (SEQ ID NO. 6)

| | | | |
|---|---|---|---|
| NGSAI_NEOTX_7 | Pancreatic | CLVS1 | RAB2A |

CAATTCCAACATGGTCATTATGCTTATTGGAAATAAAAGTGATTTAGAATCTAGAA
GAGAAGTAAAAAAAGAAGAAGGTGAAGCTTTTGCACGAGAACATGGACTCATCTT
CATGGAAACGTCTGCTAAGACTGCTTCCAATGTAGAAGAGGGCCTAATCAAGGGA
ATGGAAGATGAGGAGAATGAAGGCTCTGGGAATTTATTTTCATCGTGGACCGGAC
TTTTTATCAGCCAGGAAAAAGGTGTGGTGGCTCACGCCTGTAATCCTAGCACTTTG
GGAGGCCGAGCTGGGAGGATTTCT (SEQ ID NO. 7)

| | | | |
|---|---|---|---|
| NGSAI_NEOTX_8 | Pancreatic | CPAMD8 | NWD1 |

TGAGGTCGACGTGTGTGTGACCTCTCTTCATCTGGCCGTGACCCCCAGCATGGTCC
CCCTTGGTCGCCTGCTGGTCTTCTACGTCAGGGAGAATGGAGAAGGGGTCGCCGA
CAGCCTTCAGTTTGCAGTCGAGACCTTCTTCGAAAACCAGGTCGTTGATCTGAGGT
GGGGTATTCGGAACATTGAAGCCACTGACCACTTGACCACAGAACTCTGCTTGGA
GGAGGTTGACCGGTGTTGGAAAACATCCATAGGGCCAGCTTTTGTTGCCCTCATCG
GTGATCAGTACGG (SEQ ID NO. 8)

| | | | |
|---|---|---|---|
| NGSAI_NEOTX_9 | Pancreatic | TMEM254-AS1 | TMEM254-AS1 |

CTGGCCAATATGGTAAAACCCCATCTCTACTAAAAATACAAAAATTATCCGGGCG
TGGTGGCACGCTCCTGTAATCTCAGCTACTCAGGAGGCTGAGGACTACAGGTGCC
CGCCGCCACGGCTAGCTAATTTTTTTTTGTATTTTTTAGTAGAGACAGTGTTTCACC
GTCTCTACTAAAGATCAAGGATGGTCTTGATCTCCTGACCTGGTGATCCACCCACC
TCAGCCTCCCACAGTGCTG (SEQ ID NO. 9)

| | | | |
|---|---|---|---|
| NGSAI_NEOTX_10 | Pancreatic | LOC105375130 | PLAGL2 |

AAGTGAAGTGCCAATGTGAAATTTCGGGAACACCTTTCTCAAATGGGGAGAAGCT
GAGGCCTCACAGCCTCCCGCAACCAGAGCAGAGACCATATAGCTGCCCTCAGCTG
CACTGTGGCAAGGCTTTTGCTTCCAAATACAAGCTGTATAGGATAAACTAAACAG
GCCTCAAGAATGTGACCTCCCACGCTCCTCCATGAACAGCTCTCTCCCTGCGTCCC
AGCAACCAAAGACACTTGTTGATTTGGGAAAAACCCAGAGGAAGGATTCTGTCTG
GATTTTCTGGTACCACTGACGCATT (SEQ ID NO. 10)

| | | | |
|---|---|---|---|
| NGSAI_NEOTX_11 | Pancreatic | ANKRD27 | CPAMD8 |

AGCAGTTGCTGATGGAGATCTAGAAATGGTGCGTTACCTGTTGGAATGGACAGAG
GAGGACCTGGAGGATGCGGAGGACACTGTCAGTGCAGCAGACCCCGAATTCTGTC
ACCCGTTGTGCCAGTGCCCCAAGTGTGCCCCAGCTCAGAAGGAAACGGGACTGGT
GGTGATGACCGACCGAGTGAGCCTGAACCACCGGCAGGACGGTGGCCTCTACACC
GATGAGGCTGTCCCCGCTTTCCAGCCCCACACAGGGAGCCTGGTGGCAGTGGCTC
CTTCCAGGCACCCCCCCAGAACAGAG (SEQ ID NO. 11)

| | | | |
|---|---|---|---|
| NGSAI_NEOTX_12 | Pancreatic | IL20RB | TRIM74 |

CTCACTGCAACCTCCACCTCCTGGGTTCTAGCGTTTCTCCTGCCTCAGCCTCCCAAG
TAGCTGGGATTACAGGAATGTGCCACCATGCTTGGCTAATTTTGTATTTTTAGTAG
AGACAGTGTTTCACTATGTTGGCCAGGCTGGTCTCGAACTCCTGACCTCAGGTGAT
CCGCCCACCTTGGCCTCCCAAAGTGCTGGGATTACAGGAGTGAGCCACCACGCCC
AGCCTCCGTTGTCCTCATTTAGACTTTCCTGGGTTATAGGCACTTTTGACTTCCTGG
GGTCCTTCTTCAGTTAAAAA (SEQ ID NO. 12)

| | | | |
|---|---|---|---|
| NGSAI_NEOTX_13 | Pancreatic | CHS.26712.1 | ZNF829 |

CGGGCATGGTGGCGTGCACCTGTAGTCCCAGCTACTGAGGAGGCTGAGGCAGGAG
AATTGCTTGAACTCGGGAGGTCAAGGTTGTAGTGAGCCGAGATCGCACCACTGCA
CTCCAGCACTCCAGCCTGGGTGACAGCAAGACTCTGTCTCTGAACACAGGCCTCTA
GTCAGCTCTCTATCAACCATCCAGGGCTCTTTTCCTTGTTCCAATAAGGAGATCAC
AGCTGGCTTAGAATTGGAAAGTCCCACTGAAACCAGGTTGCTGAAATTCTCCAAC
ATCACTTCTTTGTATAAATTCATC (SEQ ID NO. 13)

| | | | |
|---|---|---|---|
| NGSAI_NEOTX_14 | Pancreatic | NA | ZNF431 |

CCTTTGAGTCTCCAGAGTCCACTGTGTCATTCTTATGCTTTTGCATCCTCATAGCTT
AGCTCCCGCTTATGAGTGAAAACATACGATGTTTGGTTTTCCATTCCTGAGTTACTT
CACTTAGAACCCTCCTTGACCTATCTCAGTGCTGGGATTACAGGCGTGAGCCACAC
CTGGCTGCCTTTTAACTGTTCTGATAAGCAAACTCTACAGTTAAAACCAATTTTTGT
GTGCACTAAAAATACCAACTTCCTCATCAAAATCTACAAAGTACC (SEQ ID NO. 14)

| | | | |
|---|---|---|---|
| NGSAI_NEOTX_15 | Pancreatic | RFX8 | RNF149 |

TCCAAAAGCAACAAGTGAAACAGATGCCAGGAGCCAAAACTATCTCTGTGGCAGA
GGGTCATGGCTTTGCTTACAGCAGTGAGAAGAAGATTCCATCTCAGCTGGAAGCT
GGCGGCCAATTTGGTAAACTCTTCTTTGCTTCTGCTTGTCTAGCAATGCCGTATTTT
CTCCTGCATCTCCTTTAAAGCTGGGATCACACTGTGGCTCAGATTCAGCAGGGGAG
GCTGATGGTGGACTGCTCTCATCACTTCCGTCATCATCTGGTAAAGCTAGACTCAA
ATTTGCAGCTGGATCCCTTCCA (SEQ ID NO. 15)

| | | | |
|---|---|---|---|
| NGSAI_NEOTX_16 | Pancreatic | KIF13B | KPNB1 |

CTGGTGTGCTCAGGGGGCCGTCCTTGTTCTGCTGCCTCTGAAGCTTCAATGGCCAA
ATCCATTTCCTCATCACAGACATTGGACCAGAATTCTATCCCTTGTAAAGCCACCT
CATCAATGTCACTTTTCATTGCTTCGATTGTGATCATTTAAGTCCAGGAATATAAC
AGGAATGTGTGTCTCCATCCCAGGTACTGGGGTCCATTCTGCTGGGGCCCCTGGAA
TACCACTGCCAGCAGAGGGGACCATCACCGCGTTCCTCTCCTCAGTTAGGGTCAAC
CGCATCTCCAGAAGCTGCGCT (SEQ ID NO. 16)

| | | | |
|---|---|---|---|
| NGSAI_NEOTX_17 | Pancreatic | RHPN2 | ZNF569 |

AAACTATAACAACTTAATCACAGTAGGCTATCCGTTCACCAAACCTGATGTGATTT
TCAAATTGGAGCAAGAAGAAGAACCATGGGTGATGGAGGAAGAAGTATTAAGGA
GACACTGGCAAGCATAATAAGAGTGCCACATACTCCGTGGGAATGCAGAAAACGT
ACTCCATGATCTGCTTAGCCATTGATGATGACGACAAAACTGATAAAACCAAGAA
AATCTCCAAGAAGCTTTCCTTCCTGAGTTGGGGCACCAACAAGAACAGACAG (SEQ
ID NO. 17)

| | | | |
|---|---|---|---|
| NGSAI_NEOTX_18 | Pancreatic | LOC105378701 | STIL |

AAACCAACTCCATTTGTCTTCCAGCTTGCACTGCGTCTTCAACAGCAGTTGTCTTA
GGGGAACAGGGCATCAGAGACTGTGCTTCCAACAAACGCTGAATCTGGTAGGATC
ATTGTGAGGCTCCAATCAGAAAGTGTCTTACACATCATACAGTAGCCCTCAGATTC
AATGTAGAAAACAGCACCAGCAAATGTAAATTAGTACAACCATTGTGGAAGACAG
TGTGG (SEQ ID NO. 18)

| | | | |
|---|---|---|---|
| NGSAI_NEOTX_19 | Pancreatic | GREB1L | LAMA3 |

GCCAGATACTGCTTCAGTTCCAGGGTTAACGTATCTCAGAATAACACGAAACAAG
GAGCCACTTGACTTCCCTACATTCAATGTTATTCTTACATCATTCTCTCCAAGAGTG
TGTTCACCATTTTTTCAAAAGTCTCGTCACATCTCAGAAGTGGGCTCGTGATCCCC
CACTGCAGAGACTTGCTGTACTCACTCAAGCCAAAGTACAGCTTCTCCTCG (SEQ
ID NO. 19)

| | | | |
|---|---|---|---|
| NGSAI_NEOTX_20 | Pancreatic | LAMC1 | LAMC2 |

GGTGCCTTCAATCTCGTTTAGCTTATTCAGGTCCACTGTATCCAGCTGCCCCAGCTG
CTCCAAGAGGTCATTAATAATGCTGAGGAGGCTAGTAACAGAGTTTTTGGCTTTTC
TGGCATTGATCTCGGCTTCTTGAGCAGCCTGTGAAGCCCATCAGATGCAGGAGGCC
GTCTAATGTGTTGAGTGTGTCTTGGATTGTAACCCCAGCGTTCTTGGCTCTGGTATC
AACCTTCTGGGCTTCTGTAATCACCATCTGTACTGCATCCATATTCGTGTCAAACTC
CAGCTCCTTCCTTTCCAG (SEQ ID NO. 20)

| | | | |
|---|---|---|---|
| NGSAI_NEOTX_21 | Pancreatic | CHEK1 | LOC105369526 |

TGAGCCTCGCCCCGGCAGCTTCCAAGAGAGAGCAGAGGTGCTGGAAAGGGCACA
AGAGCAGGAACTCGAGGACCTGGTTTGCATCTCAGCTCTGGCACGTCCTTGCTGGT
GACTCATTGCATAACCTCCCTGAGCCTTGGTCTTCTTGTCTGATTCATACAACTTTT
CTTCCATTGATAGCCCAACTTCTCACAAGTCTCTTTCAGGCATTGATAAGATTTGTC
TGCATCCAATTTGGTAAAGAATCGTGTCATTCTTTTGACCAACCGCTGCCAGGGGT
TCTGTGAGGATCCTGGGGTGC (SEQ ID NO. 21)

| | | | |
|---|---|---|---|
| NGSAI_NEOTX_22 | Pancreatic | ARHGAP32 | ME3 |

GAGGTTTAGTTTTTTTTGTTTTTTAAGTACAAGATGGAGACTGAAAGTGAGAGTAG
CACTTTAGGGGATGACAGTGTCTTCTGGTTGGAGTCTGAAGTTATAATCCAGGTGA
CTGACTGTGAAGAGGAAGAAAGGGAAGAGAAGTTCAGGGGATGGCCTTTACCCTT
GAAGAAAGGCTGCAGCTTGGAATCCACGGCCTAATCCCGCCCTGCTTTCTGAGCC
AGGACGTCCAGCTCCTCCGAATCATGAGATATTACGAGCGGCAGCAGAGTGACCT
GGACAAGTACATCATTCTCATGAC (SEQ ID NO. 22)

| | | | |
|---|---|---|---|
| NGSAI_NEOTX_23 | Pancreatic | GMNN | MYO6 |

GAGCTGTGGCCTTTTGCGAGGTGCTGCAGCCATAGCTACGTGCGTTCGCTACGAGG
ATTGAGCGTCTCCACCCATCTTCTGTGCTTCACCATCTACATAATGAATCCCAGTAT
GAAGCAGAAACAAGAAGAAATCAAAGAGAATATAAAGGTGGTTGTAATCTGAAG
AATAAATCTGCTCAGTCTTTGGAATATTGTGCTGAATTACTGGGTTTGGACCAAGA
TGATCTTCGAGTAAGTTTGACCACAAGAGTCATGCTAACAACAGCAGGGGGCACC
AAAGGAACAGTTATAAAGGTACC (SEQ ID NO. 23)

| | | | |
|---|---|---|---|
| NGSAI_NEOTX_24 | Pancreatic | CCDC134 | IRAK1 |

TCACGCCTGGCTAATTTTTGTATTTTTAGTAGAGACGGGGTTTCACCAAGCTGGCC
AAGCTGGTCTCGAACTCCCGACCTCAGGCAATCCGCCCACCTCAGCACTTTGGGA
GGCCAAGGCAGGAGGATCGCTGGAGCCCAGTAGGTCAAGACCAGCCAGGGCAAC
ATGATGAGACCCTGTCTCTGCCAAAAAATTTTTTAAACTATTAGCCTGGCGTGGTA
GCGCACGCCTGTGGTCCCAGCTGCTGGGGA (SEQ ID NO. 24)

| | | | |
|---|---|---|---|
| NGSAI_NEOTX_25 | Pancreatic | CHS.3009.1 | FAM120A |

TTTCCTGCCTTAGCCTCCCAAAGTGCTGGGATTACAGGTGTGAGGCACTATGCCCG
GTTCATTTATGTTTTAAAAGTCTCATATAACTAGCCGGGTGTGGTGGCTCATGCCT
ATAATCTCAGCATTTTGGGAGGCCAAGGAGAGAGAATTGCTTGAGGCAGGAGTTC
AAGACCAGCCTGGGCAATATAGTGAGACCCCTGCCTCTACAAAAAATTTTAAAAA
TTAGCCAGGTATGGTGGTGCACACCTGTAGTCCCAGCTACTCAGGAGGCTGAGGC
GGGAGGATCGCTAGAGCTGGGAGG (SEQ ID NO. 25)

| | | | |
|---|---|---|---|
| NGSAI_NEOTX_26 | Pancreatic | HIVEP3 | SEPHS1 |

CTATAAAATGTCCAATCACTTTCAGTTCCGTAGCAGGCTCTTCCATACTGCACACC
ATGCTTATGGCTGGAGGTCCAGTTACACATGCATGAAGGCTGCCCTGCCCACTGGT
TCCTGGAGGAGGGCGCGTCCGAGTTAAAGCCTCTTCTTAGCCTTCGGCCTTGGGAT
GGCAAACTGGTCCTGTGTTCTCTGACCCACGGATCCAGCCCCTTCTTCATGAATTA
TTCCGGCCAGGCAGGATTTTGTGCATTTTTTTCATGAACACCTGCGCGCCGGGCCG
GGGCGGCGGGAGGCGGCTTGG (SEQ ID NO. 26)

| | | | |
|---|---|---|---|
| NGSAI_NEOTX_27 | Pancreatic | JMJD1C | JMJD1C-AS1 |

AGAGCTGGTGGGTAAGCGGTTCCTGTGTGTGGCGGTCGGCGACGAGGCACGTTCG
GAGCGCTGGGAGAGCGGACGCGGCTGGCGAAGCTGGCGAGCGGGGGTCATCCGA
GCCGTGTCACACAGGGACAGCCGCAATCCGGACCTGGCGGTACTTTCAAACCTCT
GGTTGAAAGAAATATACCCAGTTCAGTCACTGCAGTAGAATTCCTTGTAGATAAG
CAACTGGATTTTTTAACTGAAGATAGTGCCTTTCAGCCCTACCA (SEQ ID NO. 27)

| | | | |
|---|---|---|---|
| NGSAI_NEOTX_28 | Pancreatic | CTSH | SMAD3 |

GGTTCAGTGCCATTTTAAATGTGTGGTTCCCATTGTTGTGGGCGTTTATCTTCCTCC
AGTTGCTGGCAAACGTCTGCAGCCTGTGGTGGTACTCCTCCGTACTGTAGGTCTTA
CGGTGCTTAGACATCCATGACTTGAAGTGAAACTTCTCCAAGTTATTATGTGCTGG
GGACATCGGATTCGGGGATAGGTTTGGAGAACCTGCGTCCATGCTGTGGTTCATCT
GGTGGTCACTGGTTTCTCCATCTTCACTCAGGTAGCCAGGGGGTGGGGTCTCTGGA
ATATTGCTCTGGGGCTCGAT (SEQ ID NO. 28)

| | | | |
|---|---|---|---|
| NGSAI_NEOTX_29 | Pancreatic | PHIP | SH3BGRL2 |

ATCCAGTTTCAGCTTTCTACATATGGCTAGTCAGTTTTTCCAGCACCACTTATTAAA
TAGGGAATCCTTTTCCCATTGCTTGTGTTTGTCAGGTTTGTCAAAGATTAGATGGTT
GTAGATGTGTGGTGTTATTTCTGAGGCCTCTGTTCTGGATTGTTTGAGCCCACGAA
TTCAAAGCCAGTCTTGTCATATTTGCTACTGGACCCAAAGCCAAAAATTAAAAGAT
GTCCATGAGAGTCTGTGCATGCAAAATGCTGACCATCAGGAGAGCATTTGCAGTC
AAATACTGCGCCATGTCCTT (SEQ ID NO. 29)

| | | | |
|---|---|---|---|
| NGSAI_NEOTX_30 | Pancreatic | LOC101929831 | NT5C3B |

ACCTTCATCACCAGAGGCTTGAAGGAACCCCGCCATGTGGCAGGGCACAGGCACT
GTTCCTGGTGAACCTTGGACCACAGCATGTCAGTGCTCTAGGGATTGTCTACTCCA
GGGATTTTCTTCAAAATTTTTAAACATGGGAAGTTCAAACCTAGTGCATAGTAGGG
AGTCAGTAAGTGTTACTCACTTCTCTCCCTTCCTCTCCTGAACCACGAGCGTTAAA
AATATTTTGTAAGGATGAAACTTCCAGAACTTGTGTTCAAATAATAATTAACACGG
GCTGGGCCTTTTCCTGAGAAGC (SEQ ID NO. 30)

| | | | |
|---|---|---|---|
| NGSAI_NEOTX_31 | Pancreatic | LOC105374140 | U2SURP |

ACTTTAAGTAAAAAGGAACAGGAAGAATTAAAGAAAAAGGAGGATGAAAAGGCA
GCTGCTGAGATTTATGAGGAGTTTCTTGCTGCTTTTGAAGGAAGTGATGGTAATAA
AGTGAAAACATTTGTGCGAGGGGGTGTTGTTAATGCAGCTAAAGGAGCACCTGTG
GGCATCTTTCCTCAACGCCCGGACTACAAATCTCTAACACGAGTTGTTGGCTGAGG
ACAGATTCTCATGGCCGGAAACCACCACTTCCCTTGGACATGCATGCGTTGGCTGG
GTACTGG (SEQ ID NO. 31)

| | | | |
|---|---|---|---|
| NGSAI_NEOTX_32 | Pancreatic | SSFA2 | UBE2E3 |

CTCCGACGCTTGCCAGGAGCTGCGGCACTTGGCCCAGGCCTTCCTCCTGCGACTCG
CCACTTGCCACTCCAGTTCCTCCTCCGCCTCCGCCGACGACGACAGGGGCCGGTCC
ATGGCCGCACTGGGGGCTCCGCTACCCCAGCCGGACCCTGCAATTAGGAGGAGGA
TCAAGGGTTATTTCAGCTAGCTCCTTCTGAATTCTTTTAGCACTAGTGGATAACTTA
GCAGTGGTTTTGCTAGAGAGTTTGGTGTTTTTCTTCTGCTGGGTGGCAGAAGGTTTT
CTTTCCTCTTGTTCTTCAGG (SEQ ID NO. 32)

| | | | |
|---|---|---|---|
| NGSAI_NEOTX_33 | Pancreatic | LOC107985961 | RP11-796E10.1 |

TCAACTCTTATCCACACAGAAGAGCTCTCTTCCAGGGCTGCTGGTGAAAGCAGGTG
CAATCAGAGGAGCCATAAGTCACAGCGATTCTGCAGGTGAGGAGGAAATGATGCC
ATGTGGCGAGACTTGGCCTTTAAGAACTGCAAATAGAGCGGAGGAGCCAAGATGG
CCGAATAGGAACAGCTCCGGTCTACAGCTCCCAGCTTGAGTGACGCAGAAGATGG
GTGATTTCTGCATTTCCATCTGAGGTACCGGGTTCATCTCACTGAATACTGCGCTTT
T (SEQ ID NO. 33)

| | | | |
|---|---|---|---|
| NGSAI_NEOTX_34 | Pancreatic | LOC400958 | TET3 |

TTGCACTAGCTGTACCAACCGCCGCACGCACCAGATCTGCAAACTGCGAAAATGT
GAGGTGCTGAAGAAAAAAGTAGGGCTTCTCAAGGAGGTGGAAATAAAGGCTGGT
GAAGGAGCCGGGCCGTGGGGACAAGGAGCGGCTGTCAAGGTGCCTCAGCCTCGA
ACCTTGTGATGAGTGAGAAATCTTTCTCCCCTACGGGTGAAGGAAAGAGCCTGAG
TCTCTGCTGTGGCTGGGGACAGGAAATGCACCCACCTGCCAAGCTGCTGGTGACA
CCTGGTGGCAGCCAGGAAGCCCCAGACT (SEQ ID NO. 34)

| | | | |
|---|---|---|---|
| NGSAI_NEOTX_35 | Pancreatic | GATA6 | SEH1L |

GATGGGGTAACTTGCTTGGGCTGAGGTTGCAGACGTTACCCCCAACAGAAGATAG
GTAGAAATGATTCCAGTGGCCTCTTTGTATTTTCTTCATTGTTGAGTAGATTTCAGG
AAATCAGGAGGTGTTTCACAATACAGAATGATGGCCTTGCCTTCCAGCTAGCAGT
ACAATGCCAATCACCACTTTCACTTTTATCCCAGACCTTAACGCTCTGATCGCTGG
AGCAGGTTGCCATCCGCCGCCCGTGGAAGTCGAAAGAGACATCGTGGATGAGATC
CTTGTGGTCCGCCGCGATGCTGC (SEQ ID NO. 35)

| | | | |
|---|---|---|---|
| NGSAI_NEOTX_36 | Pancreatic | LOC105376010 | MTAP |

GCAATATGTAATGATCTGTTTGGCTGGTGGTCACTTAATTCTTCTAACCTGTTTCCT
TATCTTTGATTGTCATTCATTTTTCCTTTTACTTTTTCTTCCATTTGTGATGCTCAGC
CACAACTTGAGATTTAAAATCATCAAAAACATACTCACCTCTCTCGTTTTGGGGCA
AAACGGCTCAGCCATTGGAATATGGCACACTCCTCTGGCACAAGAATGACTTCCA
TCATAGAAGGACTGAGGTCTCATAGTGGTCCTGTCAATGAACTGATCAATAATGA
CAATATCGCCGGGCTGAATC (SEQ ID NO. 36)

| | | | |
|---|---|---|---|
| NGSAI_NEOTX_37 | Pancreatic | CHS.27064.2 | ZG16B |

GTGAAACCCAGTCTCTACTAAAAATACAAAAATTAGCCGGGCATGGTGGTGTGCG
CCTATAATCCCAGATACTCAGGAGGCTGAGGCAGCAGAATCACTTGAACATGAGA
CGTGGAGGTTGCAGTGAGCCAAGATTGCACTACTGCACTCCAGCCTGGGTGACAG
AGTAAGACTCTGTCTAAAGAGAGAAAGAAAGAAAAGAAAAGAAAAGAGAAAAG
AAAAGAAAAGAAAAGAAAAGAAAAGAAAAGAAAAGAAAAGAAAAGGGCCAGGT
GTGGTGGCTCACACCT (SEQ ID NO. 37)

| | | | |
|---|---|---|---|
| NGSAI_NEOTX_38 | Pancreatic | CDRT1 | FGD4 |

CGGGCGCAGTGGCTCATGCCTGTAATCCCAGTACTTTGGGAGGCCGATGCGGTTG
GATCATGAGGTCAGGAGATCAAGACCATCCTGGTTAACATGGTGAAACCCCGTCT
CTACTGATACTTAGGTCATAGCTCCCGCTTAGGAGAAAGTTTTCCTCCTCACACAG
GAAGAGGGCCCGGACACTCCCAGCATGGCCTCGGAATTCAACGGGTATCGCTTTC
ACTTGTATGATGTCCAGAAGATGGATCTTTCGATTAGATGACA (SEQ ID NO. 38)

| | | | |
|---|---|---|---|
| NGSAI_NEOTX_39 | Pancreatic | MFSD12 | ZRANB3 |

GTAATCCCAGCACTTTGGGAGGCCCAGGTTGGTGGATCACCTGAGGTCAGGAGTT
CGAGACCAGCCTGGCCAGCATGGTGAAACCCCATCTCTACTAAAAATACGAAAAT
TAAGCCAGGCATGGTGTGGGGGGGGGGGGCACCTGTAATCCTCAGCCTCCCCAGT
AGCTGGGACTACAGACGCGTGCCACACCACCTGGCTAATTTTTTGTATTTTTAGTA
GAGATGGGGTTTCACTATGGTGGCCAGGCTGGTCTCAAACTCCTGAGCTCAGGC
(SEQ ID NO. 39)

| | | | |
|---|---|---|---|
| NGSAI_NEOTX_40 | Pancreatic | LAMA3 | LOC105372085 |

GTTTCTTCATATGGTGGTTACCTCACTTACCAAGCCAAGTCCTTTGGCTTGCCTGGC
GACATGGTTCTTCTGGAAAAGAAGCCGGATGTACAGCTCACTCTAGATCCACATCT
GTAAATGTCTAAGTCATGCTGCCAGCCAGTCTTGCCTACAGCTACTTGATTCTGGG
AGAGCCTTCTATAAAACTGATTACAGCATTTCCCTGCCACACAGTGAAAAAACAA
TGTAGTTTGATATGATAAAACATTGATT (SEQ ID NO. 40)

| | | | |
|---|---|---|---|
| NGSAI_NEOTX_41 | Pancreatic | PDIA4 | UBE2H |

GGGGGGCAAGTGGGGGCTTAGAGGGTGGTAGTGTGGAACACAGTTTAAAAGTCCT
GTCTCCTGTTTCTCTCCCTCCTCCCCATCCCCCCACCGTTTCCCCCTGTTGCAGGGT
TTTGTTTATATAACTCAAGTTGTTTGGCTAAATTCTTCAGATTCTTCTAACAGAGAA
AATGCCATTGAGGATGAAGAGGAGGAGGAGGAGGAAGATGATGATGAGGAAGAA
GACGACTTGGAAGTTAAGGAAGAAAATGGAGTCTTGGTCCTAAATGATGCAAACT
TTGATAATTTTGTGGCTGACAAA (SEQ ID NO. 41)

| | | | |
|---|---|---|---|
| NGSAI_NEOTX_42 | Pancreatic | MRPS18A | NA |

AAGGATATTGAGAAAAAATTACGAGGGTAGGTTTTTGAAGATGGCGGCCCTCAAG
GCTCTGGTGTCCGGCTGTGGGCGGCTTCTCCGTGGGCTACTAGCGGGCCCGGCAGC
GACCAGCTGGTCTCGGCTTCCAGCTCGCGGGTTCAGGGAAGCCTGCCGAGTGCCT
GCGATTGCAGGCACGCGCCGCCACGCCTGACTGGTTTTGGTGGAGACGGGGTTTC
GCTGTGTTGGCCGGGCGGTCTCCAGCCCCTAACCGCGAGTGATCCGCCAGCCTTGG
CCTCC (SEQ ID NO. 42)

| | | | |
|---|---|---|---|
| NGSAI_NEOTX_43 | Pancreatic | ERP44 | TEX10 |

TGCTGCAGAGCCTGCGGGTGAACAGAGTTGGGCCTGAGGAGCTGCCTGTTGTGGG
CCAGCTGCTTCGACTGCTGCTTCAGCATGCACCCCTCAGGACTCATATGTTGACCA
ATGCGATCTTGGTGCAGCAGATCATCAAGAATATCACGGTAACTTGGGTTTTTACT
CCTGTAACAACTGAAATAACAAGTCTTGATACAGAGAATATAGATGAAATTTTAA
ACAATGCTGATGTTGCTTTAGTAAATTTTTATGCTGACTGGTGTCGTTTCAGTCAGA
TGTTGCATCC (SEQ ID NO. 43)

| | | | |
|---|---|---|---|
| NGSAI_NEOTX_44 | Pancreatic | LOC101060341 | LOC284600 |

GTGGATTCCAGAGGGGTGACAGCGAAACGTGGGACCATCCAGTTGCAGGAAAAC
AAGCTTAACACGCCCACTGATTCTACATTATGGCACAGTTCACAGAGGCAGCTGCT
TTGGGAAGTTTGGTGCCAGACCCCGCCAAGCCCCTGCCCGGGGCATCTCCTCCCGC
ACCCTTCGCCGCCATCTTTCAGACGGCTGCTCTCCTGAGCCAGGCCCGCGCGCCAT
CTCCTTTAGGCTCCT (SEQ ID NO. 44)

| | | | |
|---|---|---|---|
| NGSAI_NEOTX_45 | Pancreatic | GIPR | IMPAD1 |

AATTTTTGTATTTTTAGTAGAGACGGGGCTTCACTATGTTGGTCAGGCTGGTCTTG
AACTCCTGACCTTGTGTCCTGCCTTCCTCGTCCTCCCAAAGTGCTTGGATTACAGG
CATGAGCCACTGTGCCTGGCCCCTCTTATTTTATTTTTTCGAGACAGAGTTTCACTC
TCGTTGGCCAGGCTGGAGTGCAATGGCGTGATCTCGGCTCACCGCAACCTCTGCTT
CCCGGGTTCAAGCGATTCTCCTGCCTCAGCCTCCCAAGTAG (SEQ ID NO. 45)

| | | | |
|---|---|---|---|
| NGSAI_NEOTX_46 | Pancreatic | TMEM241 | WDPCP |

GCTGGGTAGAGATCAACAGCAGTTCAAGATCTCATGTTCTTGTGTGGCTTCCTGCT
TCAGTGCTGTTTGTGGGTATAATCTATGCTGGGTCCAGAGCATTGTCCAGACTGAA
CTCCTGGGACCCTTGGACAGAGGGGATATGCTAAATGAAGCATTTATTGGCCTGTC
TTTAGCACCTCAAGGAGAAGACTCATTTCCAGATAACCTCCCTCCCTCTTGCCCAA
CCCACAGACATATTTTACAACAAAGAATACTGAATG (SEQ ID NO. 46)

| | | | |
|---|---|---|---|
| NGSAI_NEOTX_47 | Pancreatic | MUC20 | NA |

CACAGGTCTCTTTCCTCTGTCTTCCTCCATCAGGCTCCGGAAAGCTTTCCCCAGAG
AAGACGCCAGACAGCAGGGGCTGCCTCCCGGGGCTTTTGTGACCCAGCCTGTTTCT
CCATCCGAGCTGCAACCTCTGGGTGGGGGTGTCTGCACCTGCTGCATCAGCCTTTC
TGCCACTCTGGGGTCAGTGAGGTCTTCCGGGCAAGCCACACTCAGCCGCAGGAGG
AGGAAACCTCCATTTTCACCTGCACTCACGTCTGTGGTCGGCCTCGTCCGGGCAGT
CGTGGGCGTGGCTGTTGGGGGC (SEQ ID NO. 47)

### Example 5: Correlation with More Aggressive Pancreatic Cancers

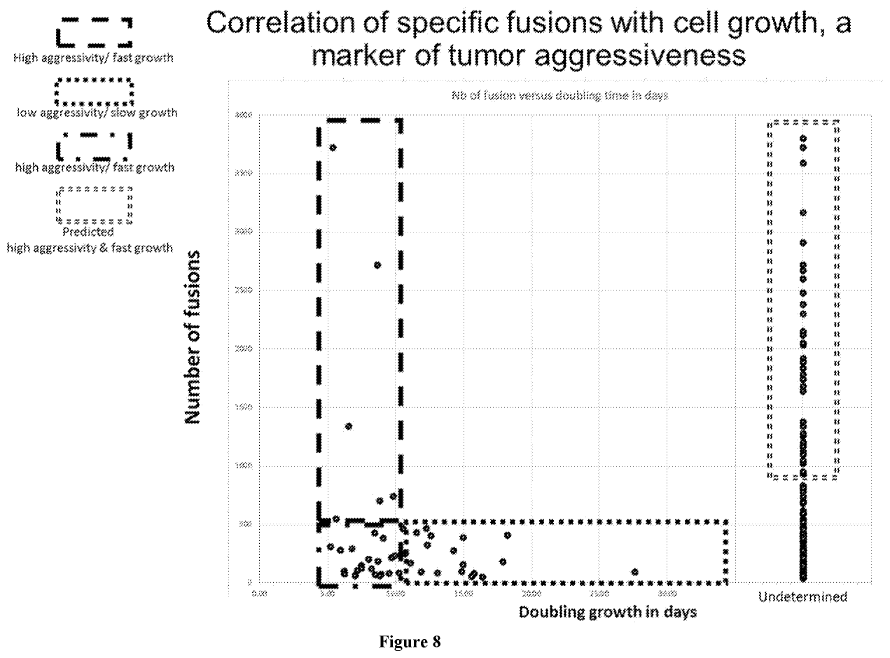

The occurrence of some pancreatic cancer-specific neotranscripts/fusions may correlate with more aggressive pancreatic cancers in terms of tumor growth. This was investigated by assessing the growth characteristics of PDX transplants as a surrogate marker of tumor aggressiveness, and by scoring their doubling time. A wide variety of growth rates was observed, as expected from the occurrence of various pancreatic cancer types and aggressiveness in the PDX collection of tumor samples (FIG. 8). The clustering of samples showing short doubling times indicated that the finding of particular sets of neotranscripts/fusions may provide an indication of the tumor progression and prognosis, thus providing useful information as whether a neoadjuvant chemotherapy may be indicated prior to a surgical resection.

### Example 6: PDX Collection Discussion

The comprehensive analysis of the DNA and RNA sequences obtained from the PDX collection, and comparison to those of normal human tissues, allowed the identification of previously unknown large genomic alterations in the tumor samples, such as gene fusions resulting from deletion, translocation, recombination, or other chromosomal rearrangement events, forming the basis of comprehensive models of cancer heterogeneity. Subsets of these neotranscripts and/or genomic alterations form a basis to generate novel diagnostic, prognostic and therapeutics analytical tools and algorithms, so as to answer unmet needs in oncology. Although pancreatic cancer is exemplified herein, it will be understood by one skilled in the art that the methods provided herein may apply to the diagnosis and prognosis of other cancer types and subtypes. Notably, neotranscripts and/or genomic alterations were identified that are associated with a plurality and/or all of known cancer-types, i.e., pan-cancer fusions (see Table 4).

TABLE 4
Pan-cancer-associated neotranscripts/gene fusions

| | Cancer | | |
|---|---|---|---|
| NGSAI_ID | Specificity | Gene1 | Gene2 |
| NGSAI_NEOTX_48 | Pan-cancer | NA | USP8 |

CTGCAGTGGACTGGGAGGCATGCCAACATGTGCTGGCATCCAAATAACATCCGCC
TCGTATATGGGTCACAGCTGAGCACGTGTTTCATGTCGTGAGTGGGCACTCCAACA
TCGCCTTGAGATTTCATCCTTTTTAAAGTAGCAGCAAGACTTTCTCCATGCAAAAA
GCAGTGCACTGACTGGGCGTGGTGCCTCACAGCTGTAATCCCAACACTCTGGGAG
ACTGAGGTGGGAGGACTGCTTGAGCCCAGGAGTTCAAGAACAGATATTTATGTTG
AGT (SEQ ID NO. 48)

| | | | |
|---|---|---|---|
| NGSAI_NEOTX_51 | Pan-cancer | NA | VPS45 |

AAAAAACTCAGTATCACTGATCATTAGAGAAATGCAAATCAAAACTATGGTGAGA
TACCATCTCAACACCAGTCAGAATGGCTATTACTAAAAAGTCAAAAAATAATAGA
TGCTGACAAGGTTGTGGAGAAAAGTGAACACTTATTCACCGTTGGTGGGAGTGTA
AATTAGTTCAACCATTGTGGAAGACAGTGTGGCAATTCATCAAAGACCTAAAGGC
AGAAATAGCATTCAACTCAGCAATCCCATTACTGGGTATATACACAACAGAATAT
AAATCATTCTATTATAAAAAGA (SEQ ID NO. 49)

| | | | |
|---|---|---|---|
| NGSAI_NEOTX_52 | Pan-cancer | LOC107987295 | NRIP1 |

TGGGCTCACTCATGCATCTGCTATCAGCTGGCTGGTTAACTGTAGTTAGTTTATCTT
GATGGCATCATTGGGGAAACTCAGCTCTCTTTCACTGGACTTCTCTTATATTTCTCC
AGCAAACTGGAAAGGGTGTGTTCTCGTGGCAGGGGCAGGAGTCCCAGGCCGCCGC
GGCTCCCAGCCTCCGGCTCCGTCAGGCTCGGTCCGCGAAGGCGCCTGCCGCCCCGT
CCTGGCCCGGCGCCCCGGCGAGCTCTTCCCTCCGACCAGCGGCGCTCACGGCGCA
GCGGCGGAC (SEQ ID NO. 50)

| | | | |
|---|---|---|---|
| NGSAI_NEOTX_53 | Pan-cancer | NA | TUBB2A |

CTCTAGGCCACCTCCTCCTCAGCCTCCTCCTCGAACTCGCCCTCCTCCTCGGCTGTG
GCATCCTGGTACTGCTGGTACTCGGACACCAGGTCATTCATGTTGCTCTCGGCCTC
GGTGAACTCCATCTCGTCCATGCCCTCGCCCGTGTACCAGTGCAGGAAGGCCTTGC
GCCGGAACATGGCCGTGAACTGCTCGGAGATGCGCTTGAACAGCTCCTGGATGGC
CGTGCTGTTGCCGATGAAGGTGGCCGACATCTTCAGGCCGCGGGGGGGGATGTCG
CACACGGCCGTCTTCACGTTGT (SEQ ID NO. 51)

| | | | |
|---|---|---|---|
| NGSAI_NEOTX_54 | Pan-cancer | LOC107987295 | NRIP1 |

CCGTGAGCGCCGCTGGTCGGAGGGAAGAGCTCGCCGGGGCGCCGGGCCAGGACG
GGGCGGCAGGCGCCTTCGCGGACCGAGCCTGACGGAGCCGGAGGCTGGGAGCCG
CGGCGGCCTGGGACTCCTGCCCCTGCCACGAGAACACACCCTTTCCAGTTTGCTGG
AGAAATATAAGAGAAGTCCAGTGAAAGAGAGCTGAGTTTCCCCAATGATGCCATC
AAGATGAACTAACTACAGTTAACCAGCCAGCTGATAGCAGATGCATGAGTG (SEQ
ID NO. 52)

| | | | |
|---|---|---|---|
| NGSAI_NEOTX_55 | Pan-cancer | LOC105379251 | NA |

GCTTAACATAACAATTTTTATTTTTATTACTTCATGTAAGAACTTCTCTACAACCAC
TGATTTTCTTACTTGCTTTCTAAGCAATGTAGAATTTTCGTCACCACTTCACCATTA
ATTTCTTGTTATTAATCCATTGTCGTTTTCCCAGCTCCAGCCTGTTAGATGAGCTCC
TGTCAACCCCAGAGTTTCAGCAAAAGGCACAACCTTTGCTAGATCCGGCGCCACT
GGGGGAGCTGAA (SEQ ID NO. 53)

| | | | |
|---|---|---|---|
| NGSAI_NEOTX_56 | Pan-cancer | WWOX | WWOX |

CAAAGGCTGCAATCACCTCAAGGCTTAACTAGGGCTGCAGAACCAACTTCGAACG
TGGTTCACTCACATGGCTGTTGGCAGGAGGCTCAGTTCTTCTACACGGGTATGCTT
GAGTATCCTCCCAACATGGCAGCTGGCTTTTCCAGCTGAGGTAGGAGAGGCTGAG
GCAGGAGAATCACTTGATCCCAGGAGGCGGAGGCTGCGGTGAGTTGAGATCACGC
CACTGCACTTCAGCCTGGGTGACAGAGCAAGACTCCATCATGGACTTGGTGAAAG
GCCTCGCCAAGGTAAACAGCAGTGT (SEQ ID NO. 54)

| | | | |
|---|---|---|---|
| NGSAI_NEOTX-57 | Pan-cancer | NA | URI1 |

TTCCAAATAGACTTTCCTTCCTCGAAACAAATCCAGAGCATCAGCAAAAGGGATCT
TATAAATGGACTTGAACCCCAACTTAAGTCCACTTAAACTTGGTGATGAGGCAAC
AATCTCCTGTTCTCGAAGAGTCTTCTCTTCATCACTTATGTTCTTTCCGGTGCTCAA
CTAAACCTACAGCCTGCTTTGCTGAGCACTTTGCAAACCAGTTGTCCCCCAGTAAA
ACAGTGACTTCATTAGTATGGACAAGTTTTCCTGGCATGAAGGCAAAAGGGC (SEQ
ID NO. 55)

| | | | |
|---|---|---|---|
| NGSAI_NEOTX_58 | Pan-cancer | CMSS1 | HP09053 |

CTGGCTTTGAGACAACGTGATTCTCCGCAGCTGGTCGCCTACCCGTGATGTTCTGC
CCACGTCGAGACCTGAGCTGAAATGGCAGACGATCTCGGAGACGAGTGGTGGGAG
AACCAGCCGACTGGAGCAGGCAGCAGCCCAGAAGCATCAGATGGTGAAGGAGAA
GGAGACACAGAAGTGATGCAGCAGGAGACAGTTCCAGTTCCTGTACCTTCAGAGA
AAACCAAACAGCCTAAAGAATGTTTTTTGATACAAC (SEQ ID NO. 56)

| | | | |
|---|---|---|---|
| NGSAI_NEOTX_59 | Pan-cancer | CRLS1 | NA |

TGGGACTACAGGCGTGTGCCACCACACCTGCCTAATTTTTTGCATTTTTTTTTTTTT
AGTAGAGACGGGGTTTCACCATGTTAGCCAGGATGGTCTTGATCTGACCTCGTGAT
CCACCCGCCTCAGCCTCTCAAAGTGCTGGGATTACAGGTGTGAGCCACTGTGCCCA
GCCACTAATTTTTTGTATTATTATTTTTTGTAGAAACAGGGTCTCACTATGTTGCCC
AGGCTGG (SEQ ID NO. 57)

| | | | |
|---|---|---|---|
| NGSAI_NEOTX_60 | Pan-cancer | FGF12 | NA |

CACTACACGCAGGCCCACGGGAATTAGATTGAAGAGAGTGTAGTCGCTGTTTTCG
TCCTTGGTCCCATCAATGGTACCATCTGGGTGCATCTGCAGGAAGTATCCCTGCTG
GCTGAATAACCTTGTCACAATCCCTTTGAGCTGGGGTTCTTTGCTCTCCATTTCGGT
CCCTTTCGAGTGCTGGGAAGTTCAATGGAAGTTGGCCGGAAGATGTGGGCCCGCT
TCAGATTCCCAAATCTGGGAAGCCAATCTGATGATTTCGCCCGTACTTCCTTCCTTC
CCCTCAGGCTTCCTTTTTTTT (SEQ ID NO. 58)

| | | | |
|---|---|---|---|
| NGSAI_NEOTX_61 | Pan-cancer | ADAP1 | SUN1 |

ACGCCGCGAGAGCCAGGTTTGAGTCCAAAGTACCCTCCTTCTACTACCGGCCCAC
GCCCTCCGACTGCCAGCTCCTTCGAGAGCAGTGGATCCGGGCCAAGTACGAGCGA
CAGGAGTTCATCTACCCGGAGAAGCAGGAGCCCTACTCGGCAGCCTGACATTTAC
CCCGGTAACTGCTGGGCATTTAAAGGCTCCCAGGGGTACCTGGTGGTGAGGCTCTC
CATGATGATCCACCCAGCCGCCTTCACTCTGGAGCACATCCC (SEQ ID NO. 59)

| | | | |
|---|---|---|---|
| NGSAI_NEOTX_64 | Pan-cancer | CMSS1 | HP09053 |

CCTGGTCTTGGTGGTATTCTCTTTTCTTTCCTTTGGTTGTATCAAAAAACATTCTTTA
GGCTGTTTGGTTTTCTCTGAAGGTACAGGAACTGGAACTGTCTCCTGCTGCATCAC
TTCTGTGTCTCCTTCTCCTTCACCATCTGATGCTTCTGGGCTGCTGCCTGCTCCAGT
CGGCTGGTTCTCCCACCACTCGTCTCCGAGATCGTCTGCCATTTCAGCTCAGGTCT
CGACGTGGGCAGAACATCACGGGTAGGCGACCAGCTGCGGAGAATCACGTTGTCT
CAAAGCCAGGCGGCCGGCG (SEQ ID NO. 60)

| | | | |
|---|---|---|---|
| NGSAI_NEOTX_65 | Pan-cancer | LOC105371307 | LOC105371308 |

CCAAATCTTATTGGATGGTTGGTATGTATCAAGGATTGTTTTACCCTCATTTAATCT
TCTCAGTAATTCAATGATTTGGAACGCTTAAAGCATTCAAAAGAATAAAATTATAG
CTTCTGCAGCAACATGGATGGAACTGGAGGCCATAATCAGGTTTGAAAATGGCTT
GTGATTCTTCCTCCATTTCAGTGTCCAACAAGCTCAGTTAGAACGTAAATGCAAGT
CCTACAGCATTCAGAGGTTCCCAAACTTTCTCAGTTTTAATGCCCTTTGTCAGAAA
TCTCTTGGTGCCCCAGCAACC (SEQ ID NO. 61)

| | | | |
|---|---|---|---|
| NGSAI_NEOTX_66 | Pan-cancer | DNAAF5 | NA |

GGGAGCCCTGAGCTTGTTTTCCTGCAACTAGACGGTCCCATGTGGGGACGATGGG
AGACAGTGACGGATCATCAGGCATTAGTTTCATAAGGAGCGTCAGCTTGGATCCC
TCGCGTGCACAGTTCACAATAGGATTTGTGCTCCTATGAGAATCTAATGCCGTTGC
CGATCTGACAGGAGGCAGAGCTCAGGTGGTAATGCTCGTTTGCCTGCCACTCACCT
CCTGCTGTGTGGCCTGGTTCCTAACAGGTCA (SEQ ID NO. 62)

| | | | |
|---|---|---|---|
| NGSAI_NEOTX_67 | Pan-cancer | LOC105371662 | LOC105371664 |

ACTTTTATAAGCTCGACTCACATGACGAAAGCCCTCATCAGATGCTTACATCATGA
TCTTGGACTTCCCAGCCTCCAGACTGATGCTATGGAAGATCAGAAAATATAAATTT
ATGAACTGCTATAAACTGTTATTTTCTTCGTGAAGATCAGACATGTGGCAGGCAAG
TTAATCTTCAGTGGAATATGCAAATAGGATTTCTGAATTTGGCATGCAAATGAATT
TGAGAGCTTCTGGGAGCATCTCTTCCAAGATTCTGGTAAGCCTTTCTTCCTGGGCG
AAACTTAGCAGAGGAAGGTAT (SEQ ID NO. 63)

| | | | |
|---|---|---|---|
| NGSAI_NEOTX_68 | Pan-cancer | NA | PTGR1 |

GGGAAGCGAGGAGCGCCTCTTCCCCGCCGCCATCCCATCTAGGAAGTGAGGAGCG
TCTCTGCCCGGCCGCCCATCGTCTGAGATGTGGGGAGCACCTCTGCCCCGCCGCCC
TGTCTGGGATGTGAGGAGCGCCTCTGCTGGGCCGCAACCCTGTCTGGGAGGTGAG
GAGCGTCTCTGCCCGGCCGCCCCGTCTGAGAAGTGAGGAAACCCTCTGCCTGGCA
ACCGCCCCGTCTGAGAAGTGAGGAGCCCCTCCGTCCGGCAGCCACCCCGTCTGGG
AAGTAGGTGGAGAGTTTTCAAACAC (SEQ ID NO. 64)

| | | | |
|---|---|---|---|
| NGSAI_NEOTX_69 | Pan-cancer | B4GALT5 | NA |

AAAAAACACAAAAATTAGCCGGGCATGGTGGCAGGTACTTGTAATCTCAGCTACT
CAGGAGGCTGAGGAAGGAGAATCGCTTGAACCCAGGAGGCAGAGGTTACAGTGA
GCTGAGATCACACGGTTGCACTCCAGCCTGGGCAACAACAGCAAAACTCCATTTC
AAAAAAACAAAGTGGCCACTGGACCAGGCACAGTGGCTCGCGCCTGTAATCCCAG
CACTTTGGGAGGTTAAGGCAGGTGGATCACCTGAAGTCAGGAGTTCGAG (SEQ ID
NO. 65)

As demonstrated hereinabove, NGS and AI-based in vitro diagnostic (IVD) assays can form a basis to better prognose tumor occurrence and evolution, and to predict the tumor response or resistance of individual patients to available therapeutics. The NGS and AI based models allowed the identification of candidate markers of tumor types and subtypes, and of some of their characteristics such as progression and response to therapeutics. These characteristics can be subjected to experimental validation and further analysis across the fields of genomics, bioinformatics, molecular/cellular biology and clinical sciences. An application of these NGS-AI models can be the pre-symptomatic detection of cancers and identification of the cancer type and subtype from non-invasive blood samples. This may lead to the prediction of its evolution and of the therapeutic response to available treatments, as well as recommendations to select the optimal treatment for each particular patient and cancer.

The identification of biological markers causally associated to tumor resistance to available treatments, by the methods disclosed herein allows early asymptomatic diagnosis as well as the preparation of efficient and specific therapeutic strategies. For example and without limitation, the identification of the genetic and epigenetic markers of tumor resistance lead to the identification and experimental validation of specific proteins that may be responsible for such resistance. Similarly, the discovery of genomic markers that allow the prediction of a pathological response or resistance to candidate therapeutics allows for patient stratification, i.e. the selection of patients that are most susceptible, e.g., to exhibit a complete pathological response upon treatment with a potential therapeutic in a clinical trial.

### Example 7: Pancreatic Sample From a Commercial Cancer Biobank

In order to test the pancreatic cancer marker gene-fusions beyond the PDX PDAC samples described herein, access to a second cohort was obtained. The majority of available cohorts are predominantly of Western origin whereas PDX PDAC collection results have a higher proportion of Asian derived ethnicity. One hundred pancreatic cancer patient derived pancreatic tissue samples of Asian genetic background were purchased from Cureline (Brisbane, CA, USA). They were made available in formalin-fixed, paraffin-embedded (FFPE) tissue and RNA extraction and sequencing was conducted on all of them. Expression and fusion discovery was done using the same approach and compared to the tables of candidates provided in Table 1, 3, and 4. In total, 4 of the gene fusion candidates were present in this 2nd cohort.

### Pancreatic Specific Set

Out of the pancreatic specific fusions, the NGSAI_NEOTX_42 (MRPS18A-NA; SEQ ID NO. 42) and NGSAI_NEOTX_47 (NA-MUC20; SEQ ID NO. 47) appeared in 20 samples and 8 samples, respectively.

### Pan-Cancer Set

The pan-cancer candidates were NGSAI_NEOTX_52 (LOC107987295, AF127936.7-NRIP1; SEQ ID NO. 50) appearing in 8 Cureline samples and NGSAI_NEOTX_61 (ADAP1-SUN1; SEQ ID NO. 59) present in 2 samples.

The fusion that contains both partners of NGSAI_NEOTX_52 was identified previously by the Peking University People's Hospital in 28 endometrial cancer patient stage III patients. (Yao et al., 2019). This study found this fusion very prevalent, in 12 out of 28 individuals and with elevated gene expression.

The fusion that contains both partners of NGSAI_NEOTX_61 was described previously in a study by the Yamaguchi University Hospital in Japan for colorectal carcinoma. (Oga et al., 2019) In this study 12 liver metastatic patients and 16 patients from a control group were analyzed. The fusion between ADAP1 and SUN1 was identified in a metastatic patient and confirmed by RT-PCR and nucleotide sequencing. This fusion pair was also found in the context of Cervical squamous cell carcinoma and endocervical adenocarcinoma (TCGA, sample DS.A7WH.01A) of a white, Latino patient.

### Example 8: Pancreatic Sample From a Public Cancer Biobank

The Genotype-Tissue Expression (GTEx) project is a comprehensive public resource to study tissue-specific gene expression and regulation. GTEx contains data from different tissue types and patients providing the opportunity to compare said data with potential non-cancer individuals (Lonsdale et al., 2013). Access to the GTEX raw sequencing data was requested and subsequently analyzed on a secure cloud platform to perform the gene fusion analysis. In total, 340 pancreatic tissue RNA-seq samples were analyzed and the results compared to the list of pancreatic cancer gene fusion candidates provided herein Table 1, 3, and 4.

### Pancreatic Specific Set

From the PDX PDAC set, the following were found in GTEX: NGSAI_NEOTX 25 (SEQ ID NO. 25), NGSAI_NEOTX_42 (SEQ ID NO. 42), and NGSAI_NEOTX_47 (SEQ ID NO. 47).

The fusion NGSAI_NEOTX_25 was observed in 1 sample of GTEX. One of its fusion partners CHS.3009.1 (see Tables 1 and 3; and identified by the Comprehensive Human Expressed SequenceS project (CHESS; led by Johns Hopkins University Center for Computational Biology) as a potential novel transcript) overlaps with the gene ENSA. Such fusions, together with the FAM120A gene as fusion partner, have not been described in the literature.

NGSAI NEOTX 42 was detected in 4 out of the 340 GTEX samples, whereas NGSAI_NEOTX_47 was found in 18 samples.

### Pan-Cancer Set

There were 3 fusion candidates from this set present in GTEX samples, NGSAI_NEOTX_52 (SEQ ID NO. 50), NGSAI_NEOTX_58 (SEQ ID NO. 56), and NGSAI_NEOTX_61 (SEQ ID NO. 59). They were present in 6, 1, and 1 cases respectively.

As described for the Cureline samples, the NGSAI_NEOTX_52 and NGSAI_NEOTX_61 fusions have been published to be clearly cancer related. The GTEX samples originated from individuals that died naturally and have donated their organs for research. None of them were diagnosed by standard cancer detection methods for which no detectable cancer was reported. It is therefore likely that a small number of GTEX pancreatic data might have been carrying an un-diagnosed cancer.

### Gene Expression Comparison GTEX Pancreatic and Pancreatic Cancer Samples

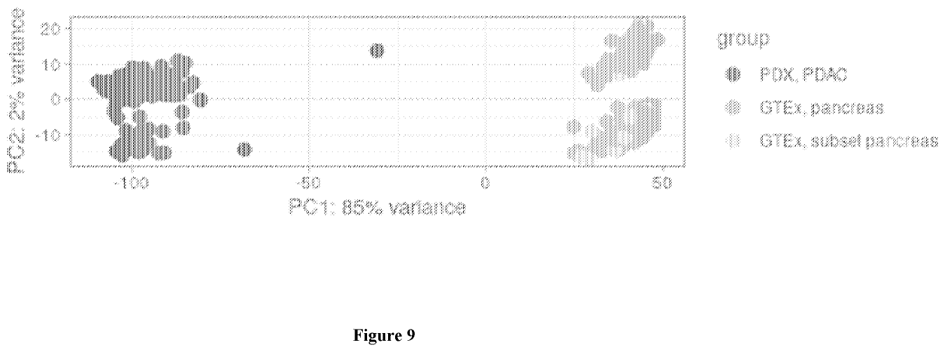

As discussed herein, some gene fusion marker candidates were detected in a subset of pancreatic GTEX samples. This raises the possibility that these GTEX samples may have undiagnosed pancreatic cancer or represent the onset of a cancer. To look into the former possibility, the gene expression profiles between GTEX pancreatic samples and the PDX PDAC cohort provided herein were compared. The focus being on the subset of pancreatic GTEX samples which contained marker fusion candidates. FIG. 9 shows the principal components analysis (PCA) of the 400 most differentially regulated genes of these samples, with the GTEX subset highlighted additionally. The GTEX subset samples clustered within the other GTEX pancreatic samples and not differently or even more closely to the pancreatic cancer samples. These individuals likely did not yet have a progressed pancreatic cancer, but it cannot be excluded that they might have had an early stage pancreatic cancer for which the prevalent gene expression changes were not yet occurring.

In view of the observations disclosed herein, the detection methods provided herein may detect early pancreatic cancer.

### Example 9: Description of Global Gene Fusion Cohort Comparison

The applied protocols for the different cohorts disclosed herein differ and subsequently pose certain limitations on the level of inter-cohort comparison. For the PDX PDAC samples all steps from tissue preparation, RNA extraction to sequencing were performed internally.

In contrast, the Cureline PDAC sample library differs in preparation and sequencing as well as the nature of the samples. Said samples were not based on fresh tissue, as in the case for the PDX samples, but slices of FFPEs. These are known to contain a higher degree of RNA degradation, leading to an increase in variations and reduced RNA fragments. This might hamper the capability to detect well expressed genes and subsequently gene fusion events in such samples, too (Williams et al., 1999).

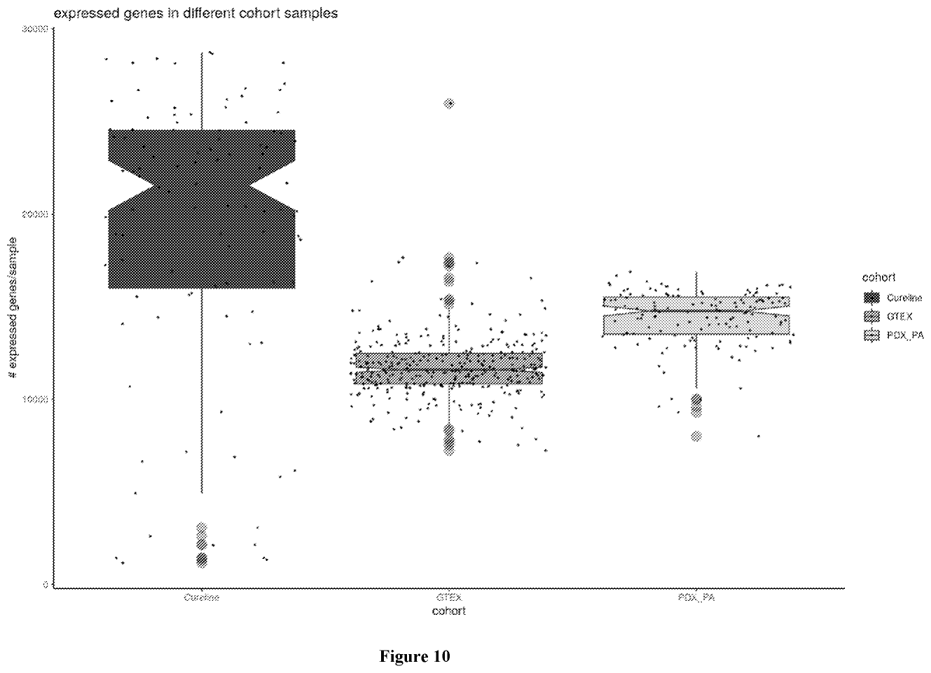

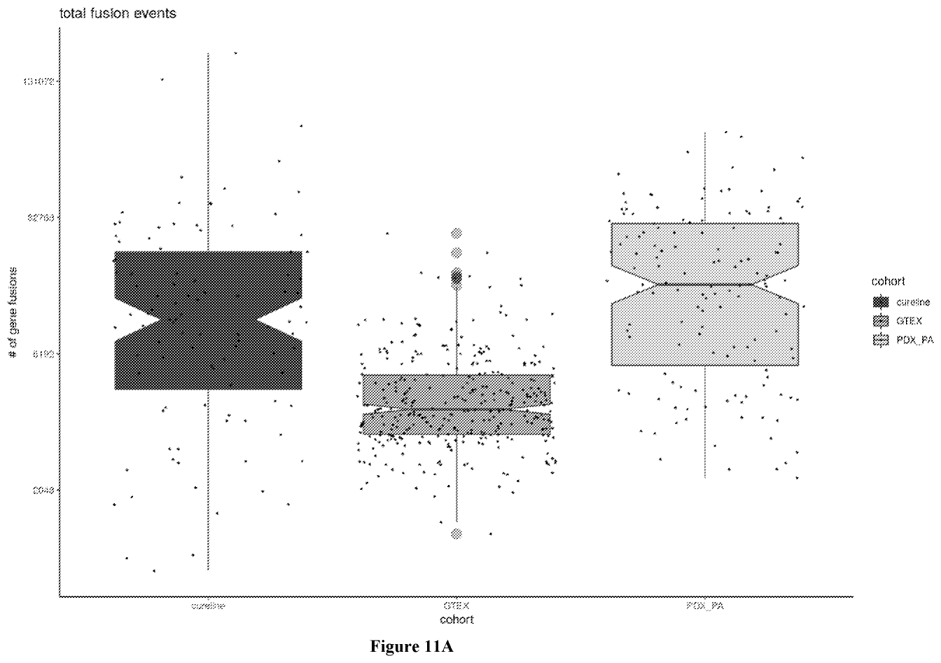

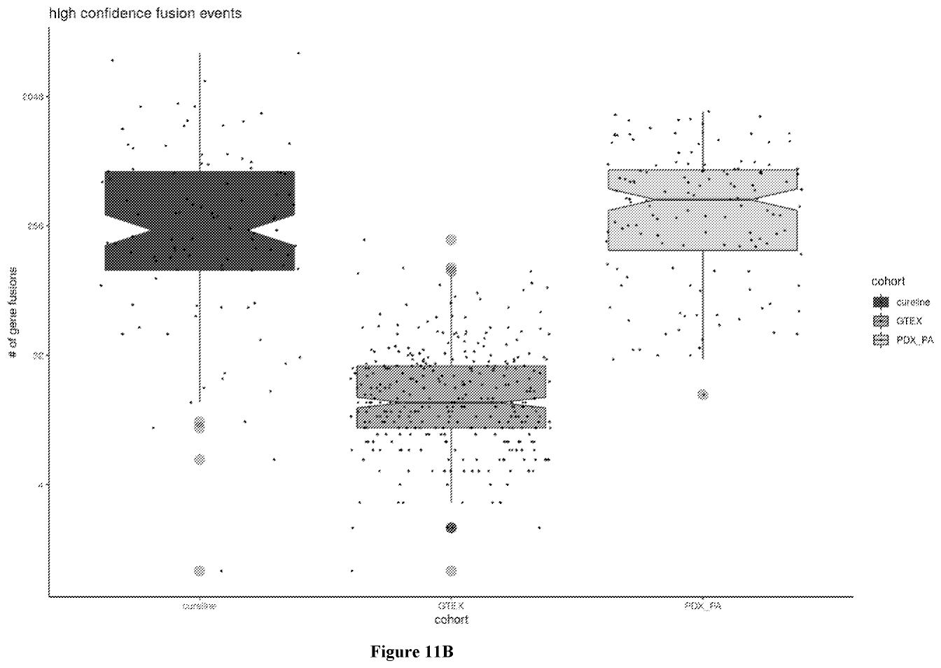

Secondly due to the nature of using a public data-set, i.e., GTEX (Genotype Tissue Expression project), control over any of the above experimental steps was not possible. To understand the impact and limitations comparison of the number of expressed genes in each of the cohorts per sample was performed (FIG. 10). It was observed that FFPE Cureline PDAC samples had an elevated number of total expressed genes. Both GTEX and PDX PDAC samples had a lower number of total expressed genes, but a more stable robust number, as assess by the expression deviation. The number of gene fusion events in these samples was compared and PDAC Cureline samples and PDAC PDX samples showed many similarities, whereas GTEX samples had a much lower number of events (FIG. 11A and FIG. 11B).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method for predicting the likelihood of progression of an asymptomatic subject to a cancerous state, comprising the steps of:
(a) sequencing at least part of the subject's genome in a sample from said subject, and
(b) identifying from the sequencing of said sample at least one gene fusion, non-gene fusion, genomic alteration, transcriptomic alteration, or neotranscript, wherein presence of said gene fusion, non-gene fusion, genomic alteration, transcriptomic alteration or neotranscript indicates an increased risk of developing cancer.

2. A method for identifying an asymptomatic subject for personalized cancer therapy, comprising the steps of:
(a) sequencing at least part of the subject's genome in a sample from said subject,
(b) identifying from the sequencing of said sample at least one gene fusion, non-gene fusion, genomic alteration, transcriptomic alteration or neotranscript, wherein presence of said gene fusion, non-gene fusion, genomic alteration, transcriptomic alteration or neotranscript identifies the subject as a candidate for personalized cancer therapy, and
(c) initiating said therapy and/or monitoring administration of the therapy to the subject.

3. A method for predicting tumor response or resistance in a subject suffering from cancer, comprising the steps of:
(a) sequencing at least part of the genome of one or more cells in a sample of the subject;
(b) identifying in said sample at least one gene fusion, non-gene fusion, genomic alteration, transcriptomic alteration or neotranscript, wherein presence of said gene fusion, non-gene fusion, genomic alteration, transcriptomic alteration or neotranscript indicates an increased risk resistant cancer.

4. A method for predicting the likelihood of metastasis in a subject suffering from cancer, comprising the steps of:
(a) sequencing at least part of the genome of one or more cells in a sample of the subject;
(b) identifying in said sample at least one gene fusion, non-gene fusion, genomic alteration, transcriptomic alteration or neotranscript, wherein presence of said gene fusion, non-gene fusion, genomic alteration, transcriptomic alteration, or neotranscript indicates an increased risk of metastasis.

5. The method of any one of claims 1 to 4, wherein the at least one gene fusion, non-gene fusion, genomic alteration, transcriptomic alteration or neotranscript is a fusion in a single gene/non-gene.

6. The method of any one of claims 1 to 4, wherein the at least one gene fusion, non-gene fusion, genomic alteration, transcriptomic alteration or neotranscript is a fusion of at least 2, 3, 4, 5, or 6 distinct chromosomal loci.

7. The method of claim 6, wherein the at least one gene fusion, non-gene fusion, genomic alteration, transcriptomic alteration or neotranscript is a fusion of at least 2 distinct chromosomal loci.

8. The method of claim 6, wherein the at least one gene fusion, non-gene fusion, genomic alteration, transcriptomic alteration or neotranscript is a fusion of at least 3 distinct chromosomal loci.

9. The method of claim 6, wherein the at least one gene fusion, non-gene fusion, genomic alteration, transcriptomic alteration or neotranscript is a fusion of at least 4 distinct chromosomal loci.

10. The method of claim 5 or 6, wherein the at least one gene fusion, non-gene fusion, genomic alteration, transcriptomic alteration or neotranscript comprises or is transcribed from at least one of the genes set forth in Table 1.

11. The method of claim 5 or 6, wherein the at least one gene fusion, non-gene fusion, genomic alteration, transcriptomic alteration or neotranscript comprises or is transcribed from at least one sequence at least 80% homologous to at least one of the provided genes set forth in Table 1.

12. The method of any one of claims 5 to 11, wherein said gene fusion, non-gene fusion, genomic alteration, transcriptomic alteration or neotranscript comprises or is transcribed from at least one sequence selected from SEQ ID Nos. 1-47.

13. The method of any one of claims 5 to 12, wherein said gene fusion, non-gene fusion, genomic alteration, transcriptomic alteration or neotranscript comprises or is transcribed from at least one sequence at least 80% homologous to a gene of SEQ ID Nos. 1-47.

14. The method of any one of claims 5 to 13, wherein the gene fusion or non-gene fusion is transcribed in a cancer cell, resulting in a transcriptomic alteration and/or the synthesis of at least one neotranscript.

15. The method of any one of claims 5 to 14, wherein the gene fusion or non-gene fusion is intra or interchromosomal.

16. The method of any one of claims 1 to 15, wherein the sample is a liquid or tissue biopsy.

17. The method of any one of claims 1 to 16, wherein the cancer is selected from: pancreatic cancer, Merkel carcinoma, Acute Myeloid Leukemia, Metastatic Carcinoma, prostate cancer, adrenal cancer, mullerian cancer, uterine cancer, kidney cancer, gall bladder cancer, cervical cancer, bladder cancer, ovarian cancer, breast cancer, head and neck cancer, esophageal cancer, lung cancer, liver cancer, colon cancer, gastrointestinal cancer, colorectal cancer, Acute lymphoblastic cancer, lymphoma, sarcoma, melanoma and brain cancer.

18. The method of any one of claims 1 to 17, wherein the cancer is pancreatic cancer.

19. A method comprising performing a bioassay to detect at least one gene fusion, non-gene fusion, genomic alteration, transcriptomic alteration, or neotranscript comprising or transcribed from at least one of the genes set forth in Table 1 in a sample from a subject, receiving the results of the bioassay into a computer system, processing the results to determine an output, presenting the output on a readable medium, wherein the output identifies therapeutic options recommended for the subject based on the presence or absence of the at least one gene fusion, non-gene fusion, genomic alteration, transcriptomic alteration, or neotranscript, wherein the sample is a is a liquid or tissue biopsy.

20. The method of claim 19, wherein the at least one gene fusion, non-gene fusion, genomic alteration, transcriptomic alteration, or neotranscript comprises or is transcribed from at least one sequence at least 80% homologous to at least one of the genes set forth in Table 1.

21. The method of claim 19 or 20, wherein the at least one gene fusion, non-gene fusion, genomic alteration, transcriptomic alteration, or neotranscript is a fusion of at least 2, 3, 4, 5, or 6 distinct chromosomal loci.

22. The method of claim 21, wherein the at least one gene fusion, non-gene fusion, genomic alteration, transcriptomic alteration or neotranscript is a fusion of at least 2 distinct chromosomal loci.

23. The method of claim 21, wherein the at least one gene fusion, non-gene fusion, genomic alteration, transcriptomic alteration or neotranscript is a fusion of at least 3 distinct chromosomal loci.

24. The method of claim 21, wherein the at least one gene fusion, non-gene fusion, genomic alteration, transcriptomic alteration or neotranscript is a fusion of at least 4 distinct chromosomal loci.

25. The method of any one of claims 19 to 21, wherein the bioassay comprises probes specific for a fusion locus comprising a sequence set forth in Table 1.

26. A cancer diagnostic kit comprising at least one reagent allowing the detection of at least one gene fusion or non-gene fusion in a sample from a subject, wherein said fusion comprises at least one gene set forth in Table 1.

27. The kit of claim 26, wherein said fusion comprises a DNA sequence at least 80% homologous to at least one of the genes set forth in Table 1.

28. The kit of any one of claims 26 to 27, wherein said fusion comprises or is transcribed from at least one sequence set forth in Table 3.

29. The kit of any one of claims 26 to 27, wherein said fusion comprises or is transcribed from at least one sequence at least 80% homologous to a gene set forth in Table 3.

30. The kit of any one of claims 26 to 29, wherein the fusion is transcribed in a cancer cell, resulting in the synthesis of at least one transcriptomic alteration, or neotranscript.

31. The kit of any one of claims 26 to 30, wherein the fusion is intra or interchromosomal.

32. The kit of any one of claims 26 to 31, wherein the kit comprises a set of probes, wherein each probe specifically hybridizes to a nucleic acid comprising the sequence set forth in set forth in Table 3.

33. The kit of any one of claims 26 to 32, wherein each probe comprises:
a nucleic acid sequence configured to specifically hybridize to the nucleic acid comprising the fusion locus, and a detectable moiety covalently bonded to the nucleic acid sequence.

34. The kit of any one of claims 26 to 31, wherein the sample is a liquid or tissue biopsy.

35. The kit of any one of claims 26 to 32, wherein the cancer is selected from: pancreatic cancer, Merkel carcinoma, Acute Myeloid Leukemia, Metastatic Carcinoma, prostate cancer, adrenal cancer, mullerian cancer, uterine cancer, kidney cancer, gall bladder cancer, cervical cancer, bladder cancer, ovarian cancer, breast cancer, head and neck cancer, esophageal cancer, lung cancer, liver cancer, colon cancer, gastrointestinal cancer, colorectal cancer, Acute lymphoblastic cancer, lymphoma, sarcoma, melanoma and brain cancer.

36. A composition comprising at least one of the following:
(a) a detection probe comprising an oligonucleotide sequence that hybridizes to a junction of a gene fusion, non-gene fusion, genomic alteration, transcriptomic alteration or neotranscript comprising at least one sequence selected from SEQ ID Nos. 1-65;
(b) a first labeled probe comprising an oligonucleotide sequence that hybridizes to a 5′ portion of a gene fusion, non-gene fusion, genomic alteration, transcriptomic alteration, or neotranscript comprising or transcribed from at least one sequence selected from SEQ ID Nos. 1-65, and a second labeled probe comprising an oligonucleotide sequence that hybridizes to the corresponding 3′ portion of the gene fusion, non-gene fusion, genomic alteration, transcriptomic alteration, or neotranscript;
(c) a first amplification oligonucleotide comprising a sequence that hybridizes to a 5′ portion of a gene fusion, non-gene fusion, genomic alteration, transcriptomic alteration, or neotranscript comprising or transcribed from at least one sequence selected from SEQ ID Nos. 1-65, and a second amplification oligonucleotide comprising a sequence that hybridizes to the corresponding 3′ portion of the gene fusion, non-gene fusion, genomic alteration, transcriptomic alteration, or neotranscript;
(d) an antibody that specifically binds to an amino acid sequence encoded by at least one sequence selected from SEQ ID Nos. 1-65; and
(e) an in situ hybridization probe for detecting a gene fusion, non-gene fusion, genomic alteration, transcriptomic alteration or neotranscript comprising at least one sequence selected from SEQ ID Nos. 1-65.

37. The composition of claim 36, wherein the gene fusion, non-gene fusion, genomic alteration, transcriptomic alteration or neotranscript is derived from a sample comprising a prostate cell or fraction, a prostatic secretion or fraction, or a combination thereof.

38. The composition of claim 36, wherein the gene fusion, non-gene fusion, genomic alteration, transcriptomic alteration or neotranscript is derived from a sample comprising a breast cell or fraction, a breast secretion or fraction, or a combination thereof.

39. The composition of claim 36, wherein the gene fusion, non-gene fusion, genomic alteration, transcriptomic alteration or neotranscript is derived from a sample comprising a pancreatic cell or fraction, a pancreatic secretion or fraction, or a combination thereof.

40. The composition of any one of claims 37 to 39, wherein the sample is a liquid or tissue biopsy.

41. The composition of claim 36 wherein the detection probe, labeled probe, in situ hybridization probe, or amplification oligonucleotide does not hybridize under stringent hybridizing conditions to DNA or RNA that is not part of, or results from, the gene fusion, non-gene fusion, genomic alteration, transcriptomic alteration, or neotranscript.

42. The composition of claim 36 wherein the first and second amplification oligonucleotides do not amplify DNA or RNA that is not part of, or results from, the gene fusion, non-gene fusion, genomic alteration, transcriptomic alteration, or neotranscript.

43. A kit comprising the composition of any one of claims 36 to 42.
